(19) United States
(12) Patent Application Publication
(10) Pub. No.: US 2014/0214060 A1
(43) Pub. Date: Jul. 31, 2014

(54) CATHETER WITH A DEPLOYABLE SCRUBBING ASSEMBLY

(71) Applicant: MEDRAD, Inc, Indianola, PA (US)

(72) Inventors: Michael J. Bonnette, Minneapolis, MN (US); Richard R. Prather, St. Michael, MN (US); David B. Morris, Anoka, MN (US); Eric J. Thor, Arden Hills, MN (US); Nicole J. Aasen, Brooklyn Park, MN (US); Debra M. Kozak, Forest Lake, MN (US); Arthur E. Uber, III, Pittsburgh, PA (US)

(21) Appl. No.: 14/115,907

(22) PCT Filed: May 18, 2012
(86) PCT No.: PCT/US12/38499

§ 371 Date: Feb. 12, 2014

Related U.S. Application Data
(60) Provisional application No. 61/487,921, filed on May 19, 2011.

(51) Int. Cl. A61B 17/3207; A61B 17/3203

(52) U.S. Cl. CPC A61B 17/3207; A61B 17/32037
USPC 606/159

(57) ABSTRACT

A catheter assembly includes an inner mandrel and an outer sheath slidably coupled along the inner mandrel. The catheter assembly includes a scrubbing assembly coupled between the inner mandrel and the outer sheath. The scrubbing assembly includes a plurality of deformable struts deflectable according to movement of the inner mandrel relative to the outer sheath. The scrubbing assembly includes a proximal shoulder coupled with the outer sheath, and a distal shoulder coupled with the inner mandrel. The plurality of struts extend between the proximal and distal shoulders. The scrubbing assembly is movable between a stored configuration and a deployed configuration according to movement of the inner mandrel relative to the outer sheath. In the stored configuration the proximal and distal shoulders are remote from each other. In the deployed configuration the proximal shoulder is engaged with the distal shoulder, and the deformable struts are deflected into bristles.

### TECHNICAL FIELD

Treatment of occluded and partially occluded vessels.

### BACKGROUND

At times thrombus in vessels matures and is sufficiently organized to resist removal through water jet thrombectomy. For instance, a water jet fails to remove appreciable thrombus from the vessel. Lytic solutions are used in some examples to assist in removal. However, in at least some examples lytics in combination with a jet of solution provide insufficient hydrodynamic force and limited dissolution of the thrombus.

In other examples cages comprising wires expanded from a catheter are used to mechanically engage and remove thrombus material from the vessel. Cages and the like are subject to radial compression through engagement with the vessel wall (or thrombus) and lack the stability to rigidly engage with the vessel wall and dislodge thrombus. Stated another way, cages are prone to deflect inwardly and fail to adequately engage and abrade thrombus material from vessel walls. The cages lack sufficient structural support for abrading engagement. For instance, the region underlying the cage at the location of engagement with thrombus is open thereby allowing the cage to readily deflect and fill the opening without abrading the thrombus. Alternatively, where cages include structural support to prevent radial compression such cages increases the risk of damage to the vessel wall through mechanical engagement that has little or no compliance. That is to say, the cages are rigid and engagement with the vessel wall accordingly abrades the wall with at least some risk of rupturing the vessel.

### BRIEF DESCRIPTION OF THE DRAWINGS

In the following detailed description, reference is made to the accompanying drawings which form a part hereof, and in which is shown by way of illustration specific examples in which the subject matter may be practiced. These examples are described in sufficient detail to enable those skilled in the art to practice the subject matter, and it is to be understood that other examples may be utilized and that structural changes may be made without departing from the scope of the present subject matter. Therefore, the following detailed description is not to be taken in a limiting sense, and the scope of the present subject matter is defined by the appended claims and their equivalents.

FIG. 1A is a perspective view of one example of a catheter including a deployable scrubbing assembly in a stored configuration.

FIG. 1B is a perspective view of the catheter of FIG. 1A with the scrubbing assembly in a deployed configuration.

FIG. 2A is a side view of the deployable scrubbing assembly of FIG. 1A in the stored configuration.

FIG. 2B is a side view of the deployable scrubbing assembly of FIG. 1A in the deployed configuration shown in FIG. 1B.

FIG. 3 is an end view of the deployable scrubbing assembly of FIG. 1A in the deployed configuration shown in FIG. 1B.

FIG. 4 is a detailed side view of the deployable scrubbing assembly shown in FIG. 1A including a plurality of deformable struts isodiametric with an outersheath and proximal and distal shoulders.

FIG. 5A is a perspective view of another example of a catheter including a plurality of deployable scrubbing assemblies in a stored configuration.

FIG. 5B is a perspective view of the catheter of FIG. 5A with the scrubbing assemblies in a deployed configuration.

FIG. 6A is a side view of the deployable scrubbing assembly of FIG. 5A in the stored configuration.

FIG. 6B is a side view of the deployable scrubbing assembly of FIG. 5A in the deployed configuration shown in FIG. 5B within a vessel.

FIG. 7 is a cross sectional view of the catheter with the deployable scrubbing assembly shown in FIG. 5A in the deployed configuration in combination with a fluid jet thrombectomy catheter.

FIG. 8A is a top view of one example of a coined retainer used to hold a deployable scrubber in a desired configuration.

FIG. 8B is a side view of the coined retainer shown in FIG. 8A.

FIG. 9A is a perspective view of another example of a gripping mechanism in a first orientation associated with the deployable scrubbing assembly in the stored configuration shown in FIG. 1A.

FIG. 9B is a perspective view of the gripping mechanism of FIG. 9A in a second orientation associated with the deployable scrubbing assembly in the deployed configuration shown in FIG. 1B.

FIG. 10A is a perspective view of one example of a gripping mechanism in a first orientation associated with the deployable scrubbing assembly in the stored configuration shown in FIG. 1A.

FIG. 10B is a perspective view of the gripping mechanism of FIG. 10A in a second orientation associated with the deployable scrubbing assembly in the deployed configuration shown in FIG. 1B.

FIG. 10C is a side view of the deployable scrubbing assembly of FIG. 1A in a cage configuration between the stored and deployed configurations.

FIG. 11A is a side view of another example of a deployable scrubbing assembly with the struts in a stored configuration including a plurality of nodes along the struts.

FIG. 11B is a side view of the deployable scrubber assembly shown in FIG. 11A rotated 90 degrees.

FIG. 12A is a perspective view of the deployable scrubbing assembly of FIG. 11A with the struts in a partially deployed configuration.

FIG. 12B is an end view of the deployable scrubbing assembly of FIG. 11A with the struts in a partially deployed configuration.

FIG. 13 is a side view of the deployable scrubbing assembly of FIG. 11A with the struts in a first intermediate configuration.

FIG. 14 is a side view of the deployable scrubbing assembly of FIG. 11A with the struts in a second intermediate configuration.

FIG. 15 is a side view of the deployable scrubbing assembly of FIG. 11A with the struts in a deployed configuration.

FIG. 16A is a side view of another example of a deployable scrubber assembly.

FIG. 16B is a side view of the deployable scrubber assembly of FIG. 16A with a distal scrubber assembly deployed.

FIG. 16C is a side view of the deployable scrubber assembly of FIG. 16A with both proximal and distal scrubber assemblies deployed.

The present subject matter may be described in terms of functional block components and various processing steps. Such functional blocks may be realized by any number of techniques, technologies, and methods configured to perform the specified functions and achieve the various results. The systems described are merely exemplary applications.

### DETAILED DESCRIPTION

In the following detailed description, reference is made to the accompanying drawings which form a part hereof, and in which is shown by way of illustration specific examples in which the subject matter may be practiced. These examples are described in sufficient detail to enable those skilled in the art to practice the subject matter, and it is to be understood that other examples may be utilized and that structural changes may be made without departing from the scope of the present subject matter. Therefore, the following detailed description is not to be taken in a limiting sense, and the scope of the present subject matter is defined by the appended claims and their equivalents.

The present subject matter may be described in terms of functional block components and various processing steps. Such functional blocks may be realized by any number of techniques, technologies, and methods configured to perform the specified functions and achieve the various results. The present subject matter may be practiced in conjunction with any number of devices, and the systems described are merely exemplary applications.

As used herein, the terms “comprises”, “comprising”, or any variation thereof, are intended to reference a non-exclusive inclusion, such that a process, method, article, composition or apparatus that comprises a list of elements does not include only those elements recited, but may also include other elements not expressly listed or inherent to such process, method, article, composition or apparatus. Other combinations and/or modifications of the above-described structures, arrangements, applications, proportions, elements, materials or components used in the practice of the present subject matter, in addition to those not specifically recited, may be varied or otherwise particularly adapted to specific environments, manufacturing specifications, design parameters or other operating requirements without departing from the general principles of the same.

In the following detailed description, reference is made to the accompanying drawings which form a part hereof, and in which is shown by way of illustration how specific embodiments of the present disclosure may be practiced. In this regard, directional terminology, such as “top,” “bottom,” “front,” “back,” “leading,” “trailing,” etc., is used with reference to the orientation of the Figure(s) being described. Because components of embodiments of the present invention can be positioned in a number of different orientations, the directional terminology is used for purposes of illustration and is in no way limiting. These embodiments are described in sufficient detail to enable those skilled in the art to practice aspects of this disclosure, and it is to be understood that other embodiments may be utilized and that structural changes may be made without departing from the scope of the present disclosure. Therefore, the following detailed description is not to be taken in a limiting sense, and the scope of the present disclosure is defined by the appended claims and their equivalents.

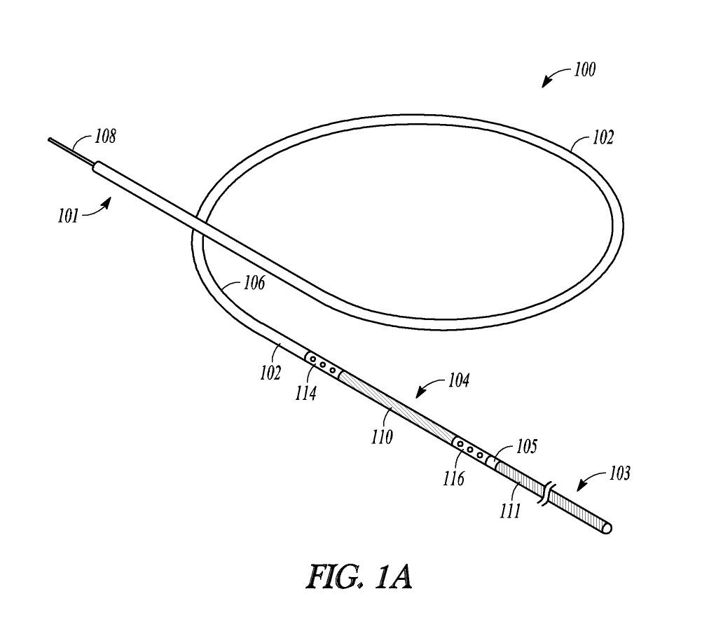

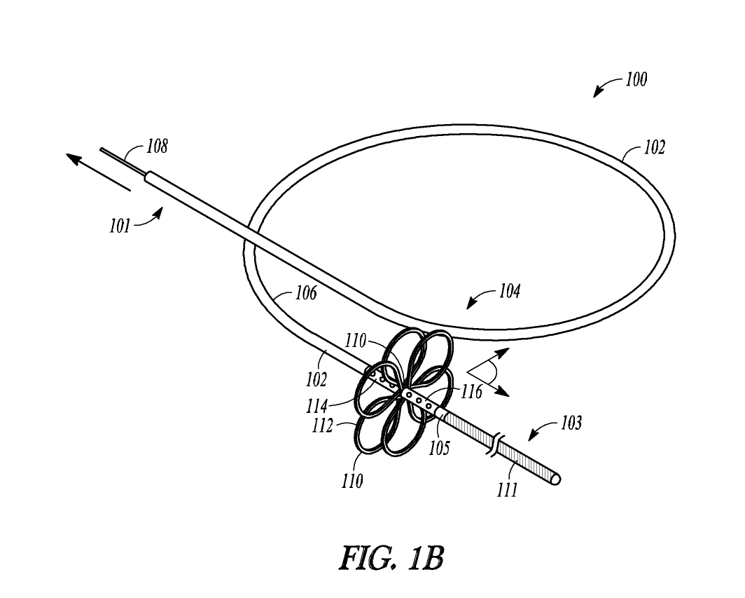

FIGS. 1A and 1B show one example of a catheter assembly 100 in stored and deployed configurations, respectively. Referring first to FIG. 1A, the catheter assembly 100 includes a catheter body 102 extending from a catheter proximal portion 101 to a catheter distal portion 103. As shown the catheter distal portion 103 includes a deployable scrubber assembly 104 deployable into a brush configuration for removal of organized thrombus within a vessel. The deployable scrubber assembly 104 will be described in further detail below. Referring again to the catheter body 102, the catheter body includes an outer sheath 106 extending from near the catheter proximal portion 101 to the catheter distal portion 103. The outer sheath 106 is coupled with the deployable scrubber assembly 104 and when used in combination with an inner mandrel 108 (described below) operates the deployable scrubber assembly 104 to deploy the scrubber assembly and thereby present a plurality of bristles as will be described herein. The inner mandrel 108 extends from the catheter proximal portion 101 to the catheter distal portion 103 through the outer sheath 106. The inner mandrel 108 is configured for slidable movement within the outer sheath 106 to actuate the deployable scrubber assembly 104.

In one example, the inner mandrel 108 is a wire including, but not limited to, stainless steel, Nitinol or a similar material extending through the outer sheath 106. In another example, the outer sheath 106 includes but is not limited to a braided material such as braided polyamide with a Teflon coating or liner on the inner diameter to facilitate sliding movement of the inner mandrel 108 relative to the outer sheath 106. In yet another example, the outer sheath 106 includes a plurality of heat shrink tubes layered over a liner configured to provide the catheter body 102. Optionally, the outer sheath 106 is constructed with a variety of materials along its length to increase or decrease the durometer of the catheter body 102 as needed, for instance to provide increased flexibility of the catheter body 102 near the catheter distal portion 103. As further shown in FIG. 1A, the catheter assembly 100 in one example includes an atraumatic tip 111 configured to provide a flexible tip to the catheter distal portion 103 and thereby facilitate the navigation of the catheter assembly 100 through vasculature. For instance, in one example the catheter assembly 100 is used as a guidewire and the atraumatic tip 111 substantially prevents the engagement of the catheter assembly 100 with a vessel wall and injury of the same. In another example, the catheter assembly 100 (similar to other examples of catheter assemblies described herein) includes a radiopaque marker 105 positioned near the deployable scrubber assembly 104 and the catheter distal portion 103 to facilitate navigation and positioning of the scrubber assembly 104 (and the catheter distal tip) at a desired location in the vasculature, for instance with fluoroscopy. One or more markers 105 are provided at any location near the catheter distal portion 103 (e.g., at each of the shoulders 114, 116) to facilitate navigation and deployment of the scrubber assembly 104. Optionally, a radiopaque coating is applied to one or more of the plurality of deformable struts 110 (described below) to assist in visualizing the struts 110 during deployment.

Referring now to FIG. 1B, the deployable scrubber assembly 104 is shown in a deployed configuration. As shown in FIGS. 1A, B the deployable scrubber assembly 104 includes a plurality of deformable struts 110. In the stored configuration shown in FIG. 1A the plurality of deformable struts 110 are tightly stored along the length of the catheter assembly 100 and thereby provide an isodiametric (e.g., near isodiametric) perimeter relative to the remainder of the catheter body 102. In one example, the catheter assembly 100 including the isodiametric deployable scrubber assembly 104 has a diameter in the range of around 0.009 to 0.045 inches. In one example, the deformable struts 110 include but are not limited to deformable materials such as superelastic materials like Nitinol, or other metallic materials such as spring steel, stainless steel, polymers, glass fiber composites, and combinations of the same. Optionally, the deformable struts include robust materials comprising, but not limited to, polymers (such as PEEK), composites and the like. In another option, the deformable struts 110 include coatings to increase the durability of the struts 110 and minimize friction between the struts and other surfaces, such as vessel walls. In yet another option, at least the struts 110 (and in some examples the shoulders 114, 116) are coated with medicaments, such as Cotavance, lysing drugs, heparin and the like. The deployable scrubber assembly 104 further includes a proximal shoulder 114 and a distal shoulder 116 with the plurality of deformable struts 110 coupled therebetween. The proximal shoulder 114 is coupled with the outer sheath 106 and the distal shoulder 116 is coupled with the inner mandrel 108.

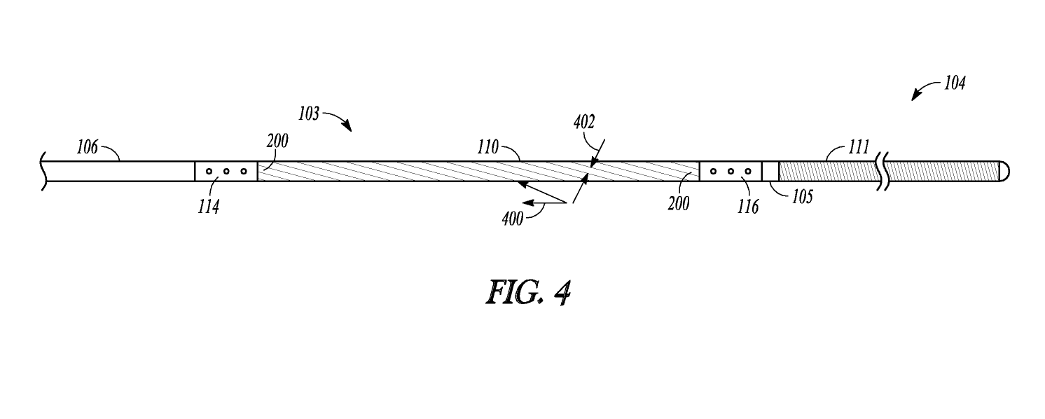

As previously described and shown in FIG. 1B movement of the inner mandrel 108 relative to the outer sheath 106 allows the inner mandrel 108 to slide within the outer sheath. Because the inner mandrel 108 is coupled with the distal shoulder 116 at the deployable scrubber assembly 104 the distal shoulder 116 is pulled toward the proximal shoulder 114 and the plurality of deformable struts 110 deflect into the deployed configuration shown in FIG. 1B. Optionally, the inner mandrel 108 is held static relative to the outer sheath 106 and the outer sheath 106 is moved distally to move the proximal shoulder 114 distally toward the distal shoulder 116 and correspondingly deploy the struts 110. In another example, where the plurality of deformable struts 110 are formed with angled cuts (see FIG. 4) rotation of the inner mandrel 108 relative to the outer sheath 106 deploys the struts 110.

As shown in FIG. 1B, one or more of the deformable struts 110 each form a bristle 112 extending from the proximal and distal shoulders 114, 116. Each of the bristles 112 is formed by a strut twisted by the movement of the shoulders 114, 116 toward each other from the remote to the adjacent positions (see FIGS. 1A, 1B respectively). As shown in FIG. 1B, the bristles 112 form a plane at an angle to the struts 110 in the stored configuration shown in FIG. 1A as well as an angle relative to the catheter distal portion 103. As shown the bristles 112 provide a two dimensional bristle (e.g. brush) shape that extends in a radial pattern away from the catheter assembly 100. As will be described in further detail below the bristles 112 of the deployable scrubber assembly 104 in the deployed configuration remove organized thrombus deposited on the walls of a vessel through reciprocating movement of the deployed bristles 112.

As shown in FIG. 1B, the proximal and distal shoulders 114, 116 are brought into engagement (e.g., through engagement of bases of each of the struts) and thereby provide a structural base for each of the bristles 112 to anchor the bristles in position on the catheter body 102 and facilitate the brushing movement of the deployable scrubbing assembly 104 within a vessel. By engaging the proximal and distal shoulders 114, 116 the shoulders (including the bases of the struts) of each of the struts provide structural stability to the deployable scrubber assembly 104 in the deployed configuration. This structural stability provided by the shoulders 114, 116 and the bristles 112 (with ends of the struts closely positioned to each other) prevents radial collapse of the bristles 112 and instead allows them to sweep back and forth in a brush like manner across organized thrombus with reciprocating movement of the catheter assembly 100. Stated another way, when reciprocated the tips of the bristles 112 are swept back and forth while the bases of the bristles, the strut bases, are anchored in place between the proximal and distal shoulders 114, 116.

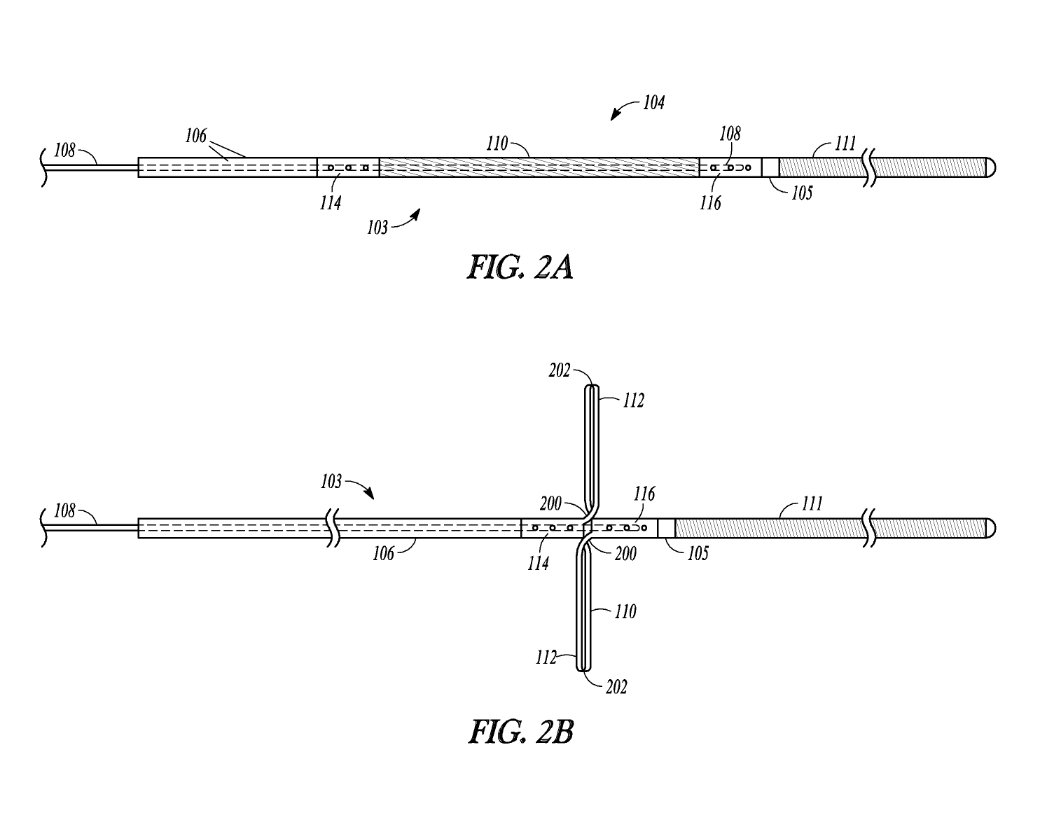

Referring now to FIGS. 2A and 2B, the deployable catheter assembly 104 is shown again in the stored and deployed configurations respectively. Referring first to FIG. 2A, the plurality of deformable struts 110 are shown in a stored configuration along the inner mandrel 108. In one example, the scrubber assembly 104 including the struts 110 has a stored diameter of around 3.3 millimeters (i.e., 0.13 inches). The inner mandrel 108 extends therein to the distal shoulder 116. As previously described, the inner mandrel 108 extends through the outer sheath 106 from the catheter proximal portion through the catheter distal portion 103 and the deployable scrubber assembly 104 to the distal shoulder 116. As shown in FIG. 2A with phantom lines the inner mandrel 108 is coupled with the distal shoulder 116. Similarly the outer sheath 106 extends from the catheter proximal portion 101 to the proximal shoulder 114 of the deployable scrubber assembly 104. The outer sheath 106 is coupled with the proximal shoulder 114 to facilitate actuation and deflection of the plurality of deformable struts 110 into the deployed configuration shown in FIG. 2B. Coupling between the inner mandrel 108 and the distal shoulder 116 as well as between the proximal shoulder 114 and the outer sheath 106 is accomplished with but not limited to adhesives, welds, mechanical fittings, crimping and the like.

As previously described, in one example the plurality of deformable struts 110 are angled relative to the outer sheath 106 and the inner mandrel 108 of the catheter body 102. The angling of the deformable struts 110 biases the struts when deflected to twist relative to the angled position shown in FIG. 2A. Stated another way, as the deformable struts 110 are deflected the struts bend along their length and at the same time rotate relative to the axis of the catheter distal portion 103 into the bristles 112 shown in FIGS. 1B, 2B. In one example the width of the struts is varied along their lengths to bias where along the strut length deflection and bending of the struts occurs. Similarly the angle of the struts 110 relative to the catheter distal portion 103 (e.g. inner mandrel 108 and outer sheath 106 at the catheter distal portion) biases the struts into twisting relative to the catheter distal portion 103 into a desired angle. By providing the struts at differing angles different configurations of bristles 112 are provided where the plane of the bristles is at one or more angles relative to the catheter distal portion 103. That is to say, by changing one or more of the angle and width of the struts tuning of the deployable scrubber assembly 104 including the bristles 112 is provided to ensure the deployable scrubber assembly 104 deploys into one or more two dimensional shapes including but not limited to a flat plane, a cup and the like. In still other examples, varying the width of the struts as well as the angle of the struts relative to the catheter distal portion 103 allows for bending and deflection of the struts into bristles having three dimensional shapes such as but not limited to a toroid, frusto-conical shapes and the like. Optionally, the struts 110 are aligned with the longitudinal axis of the catheter distal portion 103. In other words, the struts 110 have an angle measure of 0 relative to the catheter distal portion 103. As the struts 110 deflect into the bristles 112, the bristles 112 extend radially and in a substantially linear fashion from the shoulders 114, 116.

Referring now to FIG. 2B, the deployable scrubber assembly 104 is shown in a deployed configuration similar to the configuration shown in FIG. 1B. As shown in FIG. 2B the plurality of deformable struts 110 are deflected into a corresponding plurality of bristles 112 extending away from the catheter distal portion 103 (the bristles 112 otherwise extending into and out of the page are removed to expose the bristles 112 extending vertically). The configuration of the bristles 112 is two dimensional (e.g., with no appreciable thickness when compared to expanded cages) when viewed from the side and thereby provides the brush shape previously shown in FIG. 1B. In the example shown in FIG. 2B, the bristles 112 are formed to include bristle tips 202 at the outer most radius of the bristles. In one example, the scrubber assembly 104 has a deployed diameter of around 30 millimeters (i.e., 1.18 inches). The deformable struts 110 (e.g. bristles 112) extend from strut bases 200 which form portions of the shoulders 114, 116 in one example. Referring to FIG. 2B the strut bases 200 are shown engaged with the opposed shoulders 114, 116. For instance strut bases 200 associated with the proximal shoulder 114 are shown engaged with the distal shoulder 116. Similarly, the strut bases 200 associated with the distal shoulder 116 are engaged with the proximal shoulder 114.

As previously described engagement of the shoulders 114, 116 for instance with the strut bases 200 (parts of the shoulders 114, 116) therebetween provides a structurally robust base for the deployable scrubber assembly bristles 112 in the deployed configuration and ensures the bristles 112 are prevented from radially collapsing when engaged with a surface such as the inner surface of a vessel wall. The bristles 112 are in a two dimensional configuration as shown in FIG. 2B supported by the engaged shoulders 114, 116. The two dimensional configuration is not a true two dimensional plane. In other words, the bristles 112 extend along a radius, each bristle 112 has a width according to the width of the strut and some thickness. The two dimensional configuration of the bristles 112 described herein relates to the substantially planar configuration of the bristles 112 in the deployed configuration (e.g., the bristles extend in an X-Y plane cutting across the axis of the catheter with substantially no length in the Z plane). The two dimensional configuration distinguishes from other three dimensional deployed cage structures (e.g., a bulb, sphere or the like) including features that are supported between two spaced apart bases.

With the shoulders 114, 116 engaged the bristles 112 are resistant to radial collapse (as is the case with three dimensional cages with opposed and spaced apart bases). Instead, the bristles 112 described herein are capable of sweeping back and forth across organized thrombus with reciprocating movement of the catheter assembly 100 (see FIG. 1A). Stated another way, by engaging the proximal and distal shoulders 114, 116 of the deployable scrubber assembly 104 the plurality of deformable struts 110 are formed into a corresponding plurality of two dimensional bristles 112 having a strong underlying structural base that permits deflection of the bristles 112 in a backwards and forwards manner (e.g., proximally and distally) within a vessel as the catheter assembly 100 is reciprocated. The bristles sweep back and forth across organized thrombus but are otherwise incapable of collapsing radially toward the catheter distal portion 103. In contrast, cages and the like (e.g., three dimensional features with spaced apart opposed bases) of other catheter designs easily collapse under radial pressure from engagement with vessel walls because they lack structural support underlying the cages. The cages thereby fail to consistently present cutting features that reliably engage with and remove organized thrombus. By engaging the proximal and distal shoulders 114, 116 and forming two dimensional bristle shapes this sort of radial collapse is avoided and the bristles 112 instead sweep back and forth to remove this organized thrombus. In still another example, the catheter assembly 100 and the deployable scrubber assembly 104 is rotated within the vessel to provide full 360 degree engagement of the bristles 112 with the vessel walls. Optionally, the bristles 112 are distributed around the catheter assembly 100 and thereby present the scrubber assembly 104 and engage the scrubber assembly along the full perimeter of the vessel wall without rotation.

In another option, the struts 110 (and bristles 112 formed with the same) provide a mechanical support and jack much like a stent. For instance, the structural support provided by the engaged shoulders 114, 116 braces the bristles 112 and allows the bristles to dilate a vessel in a similar manner to a stent. The catheter assembly 100 (as well as the assembly 500 with multiple brushes described herein) thereby rapidly restores the flow of blood to an otherwise occluded or partially occluded vessel.

Further, the engagement between the inner mandrel 108 and the outer sheath 106 ensures the scrubbing assembly 104 remains in the deployed configuration shown in FIG. 2B during use reciprocation of the catheter assembly 100. For example, the inner mandrel 108 is retained in a position for deploying the deformable scrubber assembly 104 through one or more of frictional engagement with the outer sheath 106, mechanical fittings, crimping along the mandrel at one or more locations and engagement of the crimps within the outer sheath 106. This engagement substantially prevents sliding of the inner mandrel with corresponding collapse of the deployed scrubber assembly 104 into the configuration shown in 2A relative to the outer sheath and thereby substantially prevents the collapse of the bristles 112 in the brush shape shown in FIG. 2B. Stated another way, the engagement of the inner mandrel 108 with the outer sheath 106 substantially ensures the continued engagement between the proximal and distal shoulders 114, 116 of the deployable scrubber assembly 104. By maintaining the engagement between the shoulders 114, 116 (for instance with the strut bases 200) the bristles 112 are thereby retained in the deployed configuration shown in FIG. 2B without little risk of the bristles collapsing into an orientation similar to the stored configuration shown at FIG. 2A (where the proximal and distal shoulders 114, 116 are remote from one another). That is to say, the inner mandrel 108 and outer sheath 106 cooperate to retain the deployed brush configuration shown at FIG. 2B and thereby substantially prevent the undesired collapse of the deployed scrubber assembly 104 into the configuration shown in 2A. Optionally, the catheter assembly 110 includes other means to deploy the scrubber assembly including, but not limited to, inflatable balloons, heat activated materials such as shape memory materials and the like.

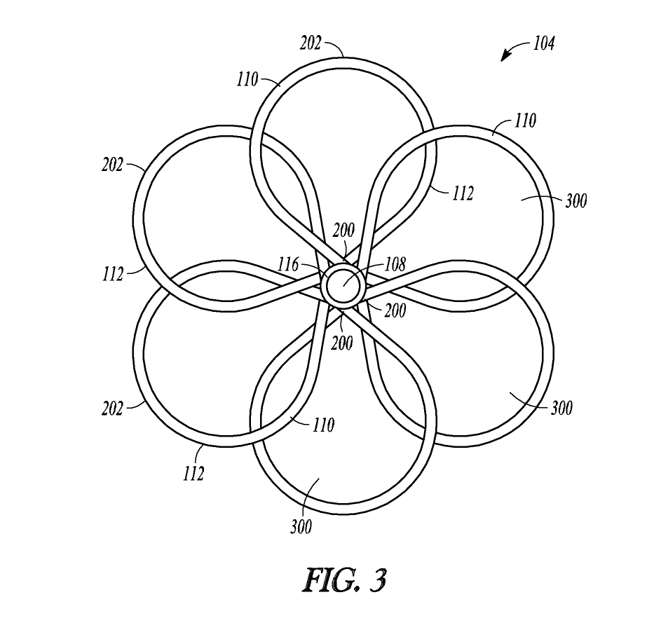

FIG. 3 shows the deployable scrubber assembly 104 in the deployed configuration corresponding to deployed configuration shown in FIG. 2B. As previously described the plurality of deformable struts 110 are formed into a corresponding plurality of bristles 112 extending from proximal and distal shoulders 114, 116. The view shown in FIG. 3 provides an axial view and thereby shows the distal shoulder of 116. The inner mandrel 108 is operated to deflect the deformable struts 110 into the deployed brush configuration shown in FIG. 3. As previously described the bristles 112 extend from strut bases 200 forming part of the proximal and distal shoulders 116 to bristle tips 202 at the outer radius of the deployed configuration. As shown in FIG. 3, the bristles 112 thereby provide a rosette configuration with each of the bristles 112 flowering away from the distal shoulder 116.

Each of the bristles 112 in the view shown in FIG. 3 provides one or more cells 300 extending from near the outer perimeter of the deployed scrubber assembly 104 to the inner perimeter of the deployed scrubber assembly adjacent to the strut bases 200. The bristles 112 are configured for deflection into the shape shown in FIG. 3 to substantially fill the entirety of a vessel the catheter assembly 100 is positioned within. By filling the entirety of the vessel the catheter assembly 100 thereby ensures reciprocating scrubbing movement of the deployable scrubber assembly 104 removes organized thrombus from around the entire vessel. Optionally, the bristles 112 are formed along an arcuate portion of the deployable scrubber assembly 104 and thereby provide a corresponding arcuate deployed configuration where the bristles 112 extend away from the proximal and distal shoulders 116 in a corresponding arc. In such a configuration the user optionally rotates the catheter assembly 100 to ensure proper engagement of the bristles 112 with the thrombus within the vessel.

Although FIG. 3 shows 6 separate bristles 112 in other examples more or fewer bristles 112 (and corresponding struts 110) are provided according to the number of cuts made into the tube used for the shoulders 114, 116 and the struts 110. One example of a cut tube used in the scrubber assembly 104 is described below and shown in FIG. 4. Further, by using more robust materials (e.g., stainless steel and the like) a tube may include a larger number of cuts with a corresponding larger number of struts 110 and bristles 112. The deployed bristles 112 (e.g, 8, 10 or more bristles) constructed with the robust material would also have adequate strength to engage and abrade thrombus where the scrubber assembly is reciprocated within a vessel. Moreover with a scrubber assembly having a greater diameter additional struts 110 are formed to maximize the number of bristles 112 when deployed.

In still another example, the plurality of deformable struts 110 are formed with nodes (described in further detail below) positioned between each of the proximal and distal shoulders 114, 116 the nodes cooperate with the proximal and distal shoulders 114, 116 to interconnect and more tightly pack the deformable struts 110 and the corresponding bristles 112 when in the deployed configuration shown in FIG. 2B. Stated another way, the cells 300 shown in FIG. 3 with nodes would be smaller and the bristles 112 would thereby be more tightly packed within the space around the catheter assembly while still filling the entirety of the vessel within which the catheter is positioned. By more tightly packing the bristles 112 a dense brush is provided with additional cutting or abrading features (bristles) for engagement with organized thrombus to thereby enhance the removal of thrombus.

FIG. 4 shows one example of the catheter distal portion 103 including the deployable scrubber assembly 104. The deployable scrubber assembly 104 is shown in the stored configuration with the plurality of deformable struts 110 in a collapsed orientation along the catheter distal portion 103. As previously described, the deployable scrubber assembly 104 includes a proximal shoulder 114 and a distal shoulder 116 with the plurality of deformable struts 110 coupled therebetween. In one example, each of the deformable struts 110 and the shoulders 114, 116 are formed from a single tube such as a tube formed with a superelastic material, such as Nitinol, heat set to assist with one or more deployment and storage of the scrubber assembly 104 in the stored configuration. Other materials for the deformable scrubber assembly 104 include but are not limited to spring steel, stainless steel and other deformable materials, polymers and the like. Optionally, the deformable scrubber assembly 104 includes an extruded polymer tube. Where the deployable scrubber assembly 104 is constructed from a single tube and multiple cuts are made along the length of the tube to form a corresponding number of the plurality of deformable struts 110. As shown in FIG. 4 in one example the plurality of cuts are made into the single tube at an angle 400 relative to the axis of the catheter distal portion 103 (e.g. including the outer sheath 106 and inner mandrel 108). The angle 400 of the cuts (e.g., struts 110) includes a range of angles, including, but not limited to, angles 400 of around −90 to +90 degrees relative to an angle of 0 aligned with the longitudinal axis of the catheter distal portion 103. In another example, the angle 400 of the cuts includes a range of angles of around −45 to +45 degrees.

As previously described, the angled struts in one example bias the struts 110 into the bristles 112 (see FIGS. 2B and 3) and a two dimensional configuration. The deflection of the plurality of deformable struts 110 according to the angle 400 thereby aligns the struts (e.g. bristles 112) in a two dimensional plane. Optionally, the deformable struts are aligned relative to the catheter distal portion (e.g., at a 0 angle or substantially close to a 0 angle) and deflect into relatively straight bristles that extend radially away from the shoulders 114, 116. In one example, the bristles in this configuration include spacing between each other. In another example, the aligned struts deflect into substantially straight bristles in a two dimensional configuration. That is to say, the bristles form narrow and straight loops extending like spokes from the shoulders with little or no overlap between the bristles. In other examples the struts 110 are cut into the single tube with one or more widths or thicknesses shown in FIG. 4 with a strut thickness 402. The varying of the strut thickness 402 alters the shape of the struts 110 when deflected into the deployed configuration. For instance at locations on the deformable struts 110 where the struts are narrow relative to other portions the struts are preferentially deflected to form bristles with corresponding bends at those narrowed portions. By altering one or more of the strut angle 400 and the strut thickness 402 the deployable scrubbing assembly 104 is configured to deploy into one or more two dimensional shapes including, but not limited to, a flat plane at an angle to the catheter distal portion 103, a cup reversible toward the catheter proximal portion 101 or toward the atraumatic tip 111 shown in FIG. 1A and the like. In other examples, altering one or more of the strut angles 400 and strut thickness 402 allows for deployable bristle configurations where the bristles form into three dimensional shapes such as a toroid, frusto-conical shapes and the like. Optionally, the single tube is cut with patterns, such as serrations, bevels and the like to provide one or more corresponding cutting surfaces on the struts.

Moreover, the struts 110 wrap (e.g., spiral) around the deployable scrubbing assembly 104 according to the angle 400 shown in FIG. 4 and the length of the cuts. The wrap of the struts 110 further controls the deployment of the struts 110 into the bristles 112, see FIG. 3. For example, the struts 110 wrap around one full circumference of the deployable scrubber assembly 104 where the cuts for each of the struts 110 have a length and angle 400 that permit wrapping of the full circumference. Stated another way, where the strut length (a hypotenuse of a right triangle) and the angle 400 are such that the sine component, the opposite side of a right triangle, is equivalent to the circumference of the deployable scrubber assembly 104 in the stored configuration, the struts 110 extend around the assembly one full rotation or 360 degrees. By increasing one or more of the angle 400 and the length of the struts 110 the wrapping of the struts 110 around the deployable scrubber assembly 104 is increased. Increasing the wrapping of the struts, for instance from half of the circumference of the deployable scrubber assembly 104 to the full circumference provides a deflectable strut that is more readily biased into the deployed configuration. Conversely, decreasing the wrapping of the struts 110 retards the deflection of the struts into the bristles 112 and may be used to stagger the deployment of struts, for instance between separate scrubber assemblies having one or more of different strut lengths or angles 400. The struts 110 wrap around the deployable scrubber assembly 104 in a range including, but not limited to, one quarter of the assembly 104 circumference to one full circumference of the assembly 104 (or 90 to 360 degrees). In another option, the struts 110 wrap around the deployable scrubber assembly greater than one circumference, for instance 450 degrees. Optionally, the struts 110 do not wrap around the deployable scrubber assembly (they are straight). The struts 110 thereby wrap around the deployable scrubber assembly in a range including, but not limited to, 0 to 450 degrees.

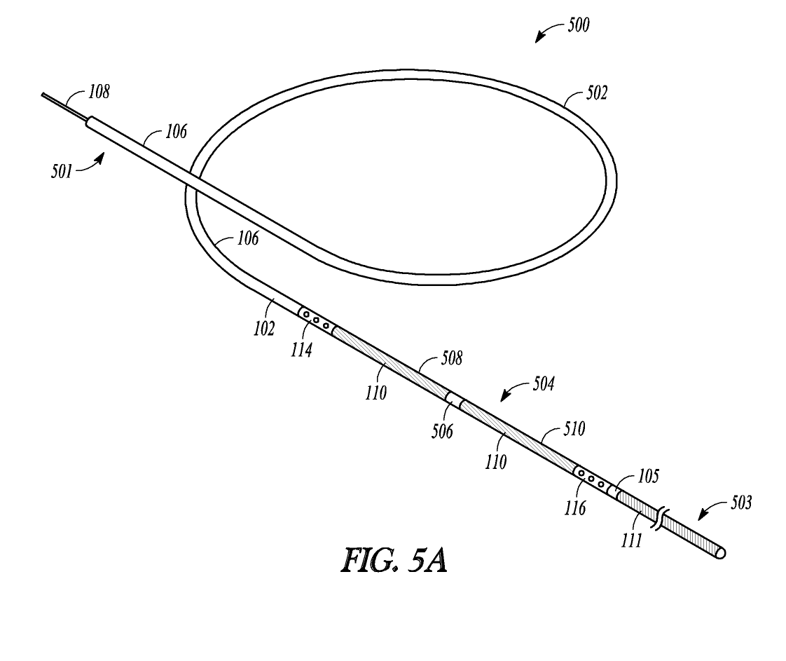

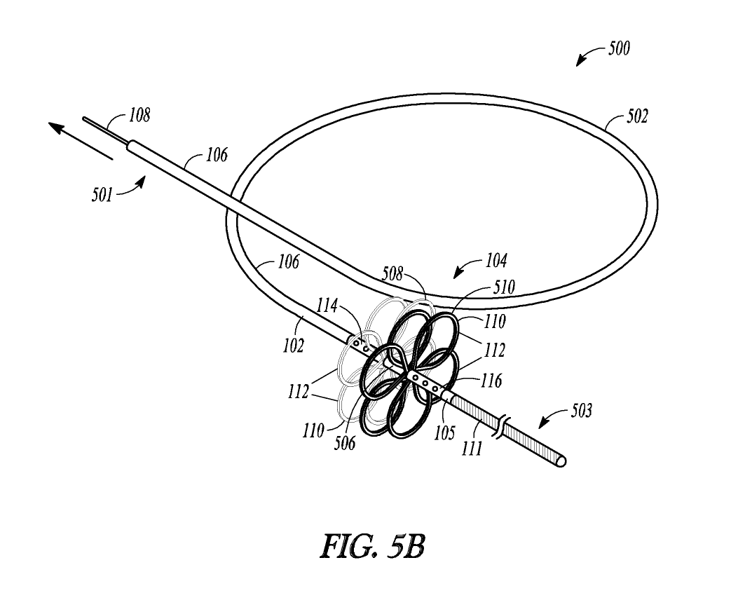

In yet another example, the struts 110 of separate scrubber assemblies (see FIGS. 5A, B below) have varying angles, lengths and wrap around the scrubber assemblies to varying degrees to tune how each of the separate scrubber assemblies deploys as described above. Optionally, the struts 110 of each of the scrubber assemblies extend clockwise or counterclockwise. In yet another option, one of the scrubber assemblies includes clockwise spiraling struts 110 while the other scrubber assembly includes counterclockwise spiraling struts 110.

Where the plurality of deformable struts 110 are cut from a single tube with the proximal shoulder and distal shoulder 114, 116 a deployable scrubbing assembly 104 is provided having a tight and compact outer perimeter relative to the remainder of the catheter assembly 104. For instance, as shown in FIG. 4 the perimeter of the catheter assembly 100 closely matches the outer perimeter of the deployable scrubber assembly 104. For example, the deployable scrubber assembly 104 has an outer perimeter that matches the outer perimeter of the outer sheath 106. This matching or flush character of the deployable scrubber assembly 104 to the remainder of the catheter assembly 100 makes the deployable scrubber assembly 104 isodiametric to the catheter assembly and assists in passage of the deployable scrubber assembly 104 through passages, vessels, delivery catheters and the like.

In contrast, other cage assembly examples use wires coupled together at one or more ends. The wires have a round shape relative to the flat character of the deformable struts 110 and are thicker than the essentially flat struts. Wires take up more room and are not isodiametric relative to the distal and proximal portions of the catheter body. Because the wires create a larger perimeter for cage assemblies they are more difficult to deliver because of their increased profile and further lack the tuning capability provided with flat struts that may have their width varied.

Referring now to FIGS. 5A and 5B, a second example of a catheter assembly 500 is shown. The catheter assembly 500 includes a deployable scrubber assembly 504 including proximal and distal brushes 508, 510. In at least some respects the catheter assembly 500 is similar to the catheter assembly 100 shown in FIGS. 1A, 1B. For instance, the catheter assembly 500 includes a catheter body 502 extending between a catheter proximal portion 501 and a catheter distal portion 503. The deployable scrubber assembly 504 is positioned at the catheter distal portion 503. Further, in the example shown in FIG. 5A the catheter assembly 500 includes an atraumatic tip 111 coupled at the catheter distal portion 503.

As shown in FIG. 5A the deployable scrubber assembly 504 includes multiple brushes, such as the proximal brush 508 and the distal brush 510, coupled in series along the catheter body 502. In the example shown the deployable scrubber assembly 504 includes a proximal shoulder 114 coupled with the proximal brush 508 and an intermediate shoulder 506 coupled between the proximal brush 508 and distal brush 510. Optionally, the intermediate shoulder 506 is considered a proximal or distal shoulder relative to the distal and proximal brushes 510, 508, respectively. A distal shoulder 116 is coupled between the distal brush 510 and the inner mandrel 108 extending through the outer sheath 106. In a similar manner, the proximal shoulder 114 is coupled with the outer sheath 106. As previously described the inner mandrel 108 is movable relative to the outer sheath 106 to actuate the deployable scrubber assembly 504 between the stored configuration shown in FIG. 5A and the deployed configuration shown in FIG. 5B.

Referring now to FIG. 5B, the deployable scrubber assembly 504 is shown in the deployed configuration with the proximal and distal brushes 508, 510 deployed relative to the catheter distal portion 503. As shown each of the proximal and distal brushes 508, 510 include a plurality of deformable struts 110 extending between the respective shoulders 114, 116, 506. Deformation of the deployable struts 110 deflects the deployable struts into the bristles 112 forming the proximal and distal brushes 508, 510. Each of the proximal and distal brushes 508, 510 deflects into a substantially two dimensional configuration extending radially away from the catheter distal portion 503. Stated another way, each of the proximal and distal brushes 508, 510 provides a brush layer to the deployable scrubber assembly 504 in contrast to the single brush shown in FIGS. 1A, 1B. Provision of multiple brushes 508, 510 (or in other examples three or more brushes and the like) enhances the ability of the scrubber assembly 504 to remove organized thrombus from within vessels.

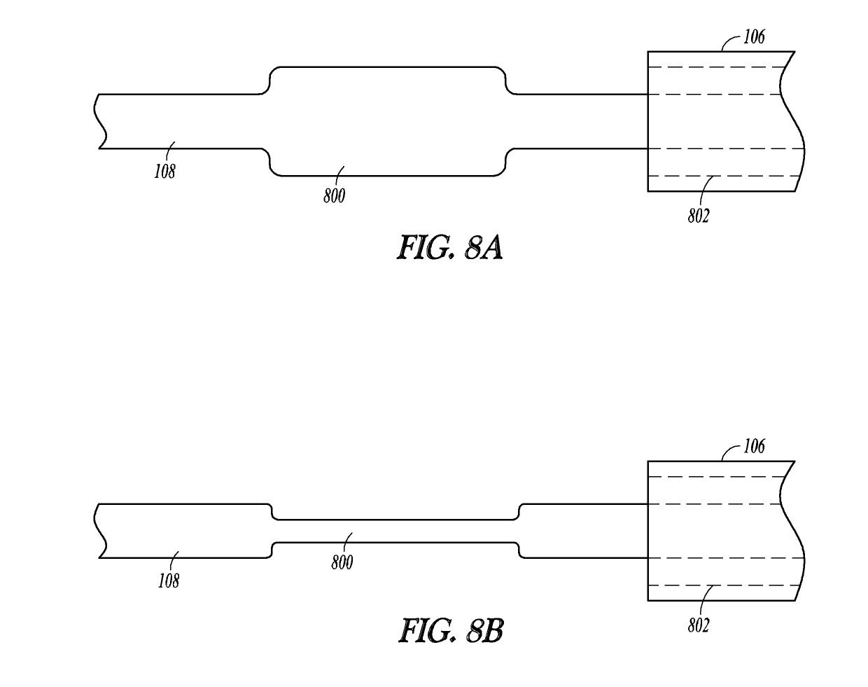

In operation, the deployable scrubber assembly 504 is moved into the deployed configuration by movement of the inner mandrel 108 proximally relative to the outer sheath 106. Movement of the inner mandrel 108 pulls the distal shoulder 116 toward the intermediate shoulder 506 and thereby engages the distal shoulder 116 with the intermediate shoulder 506. The intermediate shoulder 506 is pulled toward the proximal shoulder 114 coupled with the proximal brush 508. Movement of the shoulders 114, 116, 506 from the remote positions shown in FIG. 5A to the adjacent positions shown in FIG. 5B deflects the deformable struts 110 into the deployed configuration. Optionally, a retaining feature is included in the catheter assembly 500 that holds the intermediate shoulder 506 in place and allows for the selective deployment of one of the brushes 508, 510. For instance, a stylet or the like is engaged with the intermediate shoulder and distal movement of the outer sheath 106 deploys the proximal brush 508 while proximal movement of the inner mandrel 108 deploys the distal brush 510, respectively. In another example, a feature such as a coin 800 (shown in FIGS. 8A, B) is provided on the inner mandrel 108 for selective engagement with the inner perimeter of the intermediate shoulder 506. Engagement of the coin 800 thereby allows for individual deployment of either of the proximal and distal brushes 508, 510 through operation of the inner mandrel 108 and the outer sheath 106, respectively. In one example, the proximal shoulder 114 is moved distally to deploy the proximal brush 508. In another example, the intermediate shoulder 506 is moved proximally (e.g., through engagement of the coin 800 with the shoulder) to deploy the proximal brush 508. The position of the coin 800 along the inner mandrel 108 relative to the intermediate shoulder 506 will determine how the proximal and distal brushes 508, 510 are staggered in their deployment, their order of deployment and the degree of deployment of one of the brushes before the other brush is deployed.

The deployable scrubber assembly 504 including the deformable struts 110 (and shoulders 114,116, 506) in one example is formed with, but not limited to, superelastic materials including Nitinol and the like. In other examples the deployable scrubber assembly 504 including for instance the shoulders 114, 116, 506 and the deformable struts includes materials such as spring steel, stainless steel and the like.

As with the previous catheter assembly 100, in one example the catheter assembly 500 includes the plurality of deformable struts 110 at an angle relative to the catheter distal portion of 503. Each of the bristles 112 is formed by a corresponding strut 110. When the shoulders 114, 116, 506 are pulled into the adjacent configuration show in FIG. 5B each of the struts 110 is twisted into a bristle 112 at an angle relative to the catheter distal portion 503. As shown in FIG. 5B, the bristles 112 form a two dimensional plane at an angle relative to the struts 110 in the stored configuration (FIG. 5A) as well as the catheter distal portion 503. Actuation of the inner mandrel 508 relative to the outer sheath 506 deflects each of the deformable struts 110 into the bristles 112. Further movement of the inner mandrel 108 relative to the outer sheath 506 moves each of the shoulders 114, 116, 506 into engagement (for instance through strut bases) with each other and thereby provides a strong structural base for each of the bristles 112. By providing this structural stability to each of the bristles 112 radial collapse of the bristles 112 is substantially prevented. The structural support provided by the engagement of the shoulders 114, 116, 506 allows the bristles 112 to sweep back and forth and engage with organized thrombus within the vessel by reciprocating movement of the catheter assembly 500. Stated another way, because of the engagement of the shoulders 114, 116, 506 (including the bases of the struts) the bristles 112 are maintained in the deployed configuration shown in FIG. 5B throughout reciprocation of the catheter assembly 500 and collapse of the deployable scrubber assembly 504 including the proximal and distal brushes 508, 510 into the configuration of FIG. 5A is thereby avoided until desired by the user (e.g. through movement of the inner mandrel 108 distally relative to the outer sheath 106).

In another example the provision of multiple brushes such as the proximal and distal brushes 508, 510 provides a centering feature for the catheter distal portion 503. As shown in FIG. 5B both the brushes 508, 510 provide structural bristles 112 extending away from the catheter distal portion 503. The brushes 508, 510 extend radially away from the catheter distal portion 503 and upon engagement with vessel walls center the catheter distal portion within the vessel. That is to say, the proximal and distal brushes 508, 510 provide spaced apart supports having similar diameters that center the catheter distal portion 503 within the corresponding portion of vasculature the distal portion is delivered to. Centering of the catheter distal portion 503 assists in some examples with additional procedures within the vasculature. For instance, where the catheter assembly 500 is used as a guide wire the deployable scrubber assembly 504 in the deployed configuration centers instruments delivered along the catheter assembly 500 to the catheter distal portion 503. As will be described in further detail below, in one example a thrombectomy catheter is delivered along the catheter assembly 500 to the catheter distal portion 503. Operation of the deployable scrubber assembly 504 centers the catheter distal portion 503 and correspondingly centers the thrombectomy catheter at the distal portion 503.

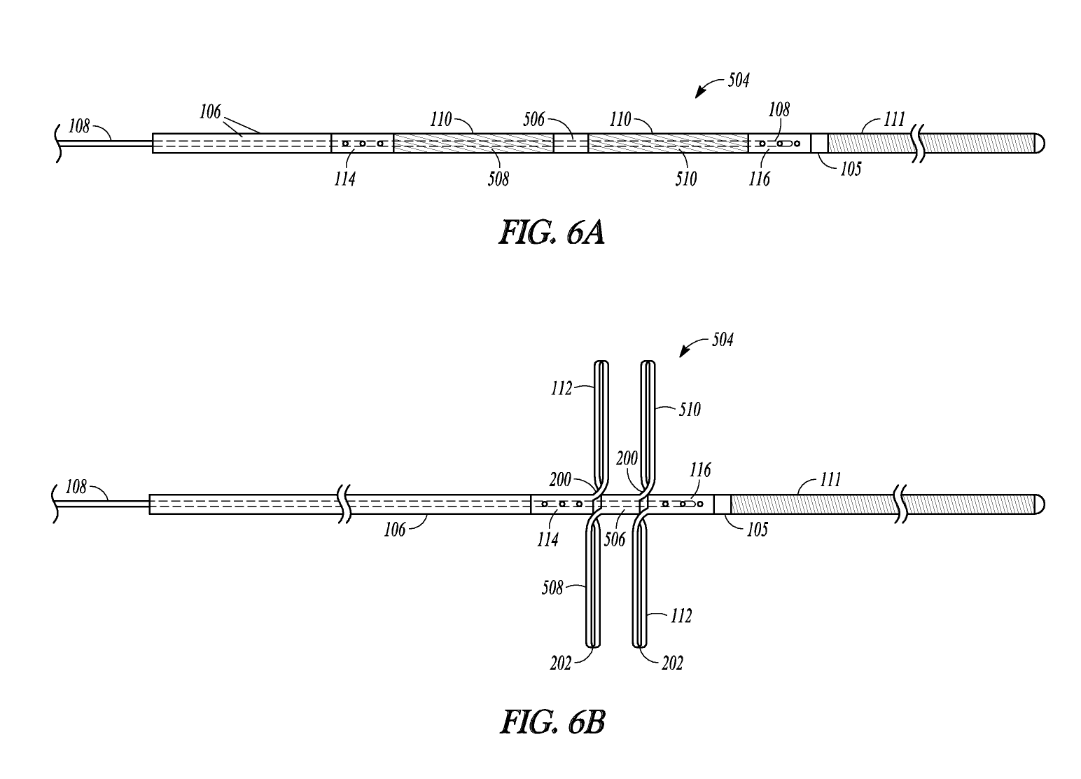

FIGS. 6A and 6B show the catheter distal portion 503 including the deployable scrubber assembly 504 in stored and deployed configurations, respectively. Referring first to FIG. 6A the deployable scrubber assembly 504 is shown with the proximal and distal brushes 508, 510 in the stored configuration where the plurality of deformable struts 110 are positioned along the catheter body 502 (e.g. the deformable struts 110 are substantially coincident with the inner mandrel 108 and the outer sheath 106). As shown in FIG. 6A the inner mandrel 108 extends through each of the proximal and distal brushes 508, 510 to the distal shoulder 116 and is coupled at the distal shoulder 116 with the deployable scrubber assembly 504. Stated another way, the inner mandrel 108 is slidably coupled with the plurality of deformable struts 110, the intermediate shoulder 506 and the proximal shoulder 114 while being coupled (e.g., fixed) with the distal shoulder 116. The outer sheath 106 extends to the proximal shoulder 114 and is coupled with the proximal shoulder. As shown in FIG. 6A the intermediate shoulder 506 is interposed between the plurality of deformable struts 110 forming the proximal and distal brushes 508, 510. Stated another way, the intermediate shoulder 506 serves as a distal shoulder to the proximal brush 508 and a proximal shoulder to the distal brush 510. Interposing the intermediate shoulder 506 between the proximal and distal brushes 508, 510 separates the brushes 508, 510 and facilitates the deployment of separate brushes as shown in FIG. 6B. Stated another way the intermediate shoulder 506 serves as an anchor point in a similar manner to the proximal and distal shoulders 114, 116 and thereby serves as a base for the bristles 112 of each of the proximal and distal brushes 508, 510.

Referring again to FIG. 6A, as shown the deployable scrubber assembly 504 in the stored configuration includes a plurality of deformable struts 110 that are substantially isodiametric with the outer sheath 106. In one example where the outer sheath 106 extends to the proximal shoulder 114 and has a substantially similar diameter to the plurality of deformable struts 110 (as well as the proximal, distal, and intermediate shoulders 114, 116, 506) the deployable scrubber assembly 504 presents an identical or near identical profile to the outer sheath and thereby facilitates the passage of the deployable scrubber assembly 504 for instance through a delivery catheter, vasculature and the like. As previously described above, where the deployable scrubber assembly 504 is constructed with a single tube with one or more cuts or slots to define each of the deformable struts 110 the deployable scrubber assembly 504 provides a compact profile that closely matches that of the outer sheath 106 and the atraumatic tip 111. In contrast, where other cage assemblies use separate wires coupled at their ends the wires generally have circular or non-flat geometries and thereby provide a larger profile relative to the remainder of the catheter and are not otherwise isodiametric with features the outer surface of the catheter including the cage assembly.

Referring now to FIG. 6B the deployable catheter assembly 504 is shown in the deployed configuration with each of the proximal and distal brushes 508, 510 deployed with bristles 112 extending away from the proximal, intermediate and distal shoulders 114, 506, 116. As shown each of the bristles 112 presents a bristle tip 202 extending from strut bases 200. In contrast to the single scrubber design previously shown in FIGS. 1A and 1B the multiple scrubber assembly shown in FIGS. 6A and 6B provides a multi-layered brush (for instance including multiple proximal and distal brushes 508, 510) that presents multiple brushes 508, 510 capable of engaging and removing thrombus within a vessel. The multi-layered brushes 508, 510 enhance the ability of the deployable scrubber assembly 504 to remove organized thrombus from the vessel. That is to say, by providing additional layers to the deployable scrubber assembly 504 the effectiveness of the deployable scrubber assembly is enhanced.

As previously described with the deployable scrubber assembly 104 shown in FIGS. 2A and 2B, the deployable scrubber assembly 504 shown in FIG. 6B engages the shoulders 114, 506, 116 to provide stable bases for each of the bristles 112. For instance the proximal, intermediate and distal shoulders 114, 506, 116 are engaged with strut bases 200 forming part of the shoulders. As shown in FIG. 6B the strut bases 200 are engaged with the opposed shoulder, for instance the strut base coupled with the proximal shoulder 114 is directly engaged with the intermediate shoulder 506. Similarly the strut bases 200 associated with the intermediate shoulder 506 are correspondingly engaged with the proximal and distal shoulders 114, 116. The stability provided by the engagement of the shoulders 114, 116, 506 substantially prevents the radial collapse of the bristles 112 when engaged for instance with organized thrombus along a vessel wall and the like. Instead, reciprocating movement of the catheter assembly 500 relative to organized thrombus sweeps the bristles 112 back and forth across the thrombus to abrade and dislodge the thrombus. The bristles 112 are not otherwise subject to radial collapse until storage of the brushes 508, 510 is desired.

In another example, one or both of the scrubber assemblies 104, 504 are operated with mechanically driven reciprocation (e.g., oscillation) or vibration to assist in removal of thrombus. For instance, one or more of the inner mandrel 108 and the outer sheath 106 are coupled with a reciprocation or vibration transmitting assembly that transmits one or more of corresponding reciprocating and vibratory motion to the scrubbing assemblies 104, 504. In still another example, the catheter assembly (e.g., the outer sheath 106) is navigated in cooperation with selective deployment and storage of the scrubbing assemblies 104, 504 to navigate the atraumatic tip 111 (see FIG. 6B) through tortuous vasculature in an inch worm or snake like fashion.

Further, as previously described the multiple brushes 508, 510 of the deployable scrubber assembly 504 provide a centering function for the catheter assembly 500. For instance, the bristles 112 extend away from the shoulders 114, 116, 506 in a radial two dimensional pattern having substantially identical shapes. Because the brushes 508, 510 are axially spaced apart the radially extending bristles 112 provide supports that center the catheter distal portion 503 within the corresponding portion of the vessel. Using the deployable scrubber assembly 504 the centering function facilitates the delivery and use of other instruments adjacent to the deployable scrubber assembly near the center of the vessel and correspondingly prevents engagement of such instruments along the vessel wall.

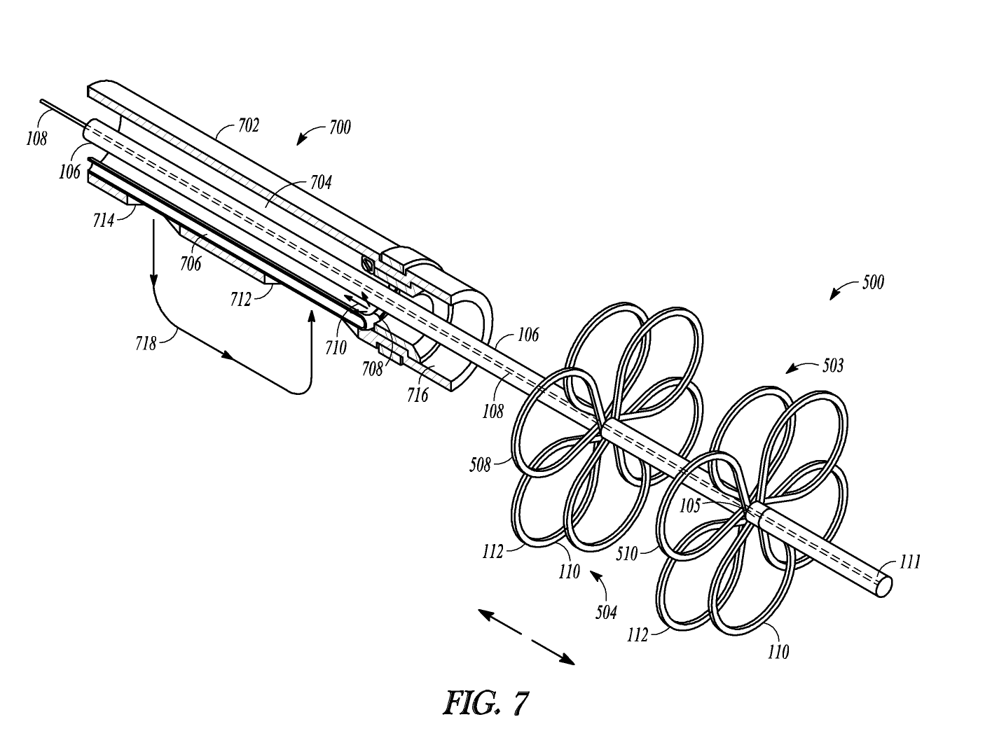

FIG. 7 shows one example of a thrombectomy catheter 700 used in combination with the catheter assembly 500 including the deployable scrubber assembly 504. As shown in FIG. 7, the thrombectomy catheter 700 includes a thrombectomy catheter proximal portion 702 shown in detail for discussion purposes. The thrombectomy catheter 700 includes a catheter lumen 704 extending through the thrombectomy catheter to facilitate the delivery of an instrument, such as the catheter assembly 500. The thrombectomy catheter 700 further includes a hypo tube 706 extending within the catheter lumen 704 to a fluid jet emanator 708. The fluid jet emanator 708 includes one or more fluid jets 710 (e.g. orifices along one or more surfaces of the fluid jet emanator 708) that provide a directional fluid flow within the thrombectomy catheter to create a circular flow as described in further detail below. The hypo tube 706 is configured to provide a high pressure flow of fluid to the fluid jet emanator 708 to generate the fluid jet 710.

The thrombectomy catheter 700 further includes one or more inflow orifices 712 and outflow orifices 714 extending through the wall of the thrombectomy catheter 700. As shown in FIG. 7, the inflow and outflow orifices 712, 714 cooperate with the fluid jets 710 from the fluid jet emanator 708 to develop a circular flow 718 beginning at the outflow orifice 714 that moves distally along the thrombectomy catheter 700 toward the inflow orifice 712 where the flow of fluid returns to the catheter lumen 704. The circular flow 718 impinges against thrombus along vessel walls, breaks up the thrombus and draws it into the thrombectomy catheter 700. The flow of fluid within the catheter lumen 704 thereafter moves the removed thrombus along the catheter lumen 704 to an exhaust at the distal portion of the thrombectomy catheter 700.

The thrombectomy catheter 700 further includes a distal opening 716. In the example shown in FIG. 7 the distal opening 716 is an aperture at the tip of the thrombectomy catheter 700. The distal opening 716 facilitates the delivery of the catheter assembly 500 through the thrombectomy catheter distal portion 702. In the example shown in FIG. 7 a double brush assembly including the brushes 508, 510 is used and shown in the deployed configuration. In another example the single scrubber assembly 104 used with the catheter assembly 100 is used in combination with the thrombectomy catheter 700.

As previously described, the catheter assembly 500 used in combination with the thrombectomy catheter 700 includes two or more brushes 508, 510. The two or more brushes 508, 510 are deployed from the catheter assembly 500 through movement of the inner mandrel 108 relative to the outer sheath 106. Movement of the mandrel relative to the sheath deflects a plurality of deformable struts 110 into bristles 112 as shown in FIG. 7. Reciprocating movement of the catheter assembly 500 (optionally in combination with movement of the thrombectomy catheter 700) is used to correspondingly reciprocate the brushes 508, 510 relative to organized thrombus and abrade the organized thrombus from the vessel wall. As shown in FIG. 7, for instance each of the bristles 112 is sized and shaped for reciprocating back and forth movement within a vessel to engage with and remove organized thrombus along the vessel wall.

In operation, the thrombectomy catheter 700 acts as a delivery catheter for the catheter assembly 500. For instance, in one example the thrombectomy catheter 700 is delivered into the vasculature along a guide wire extending through the catheter lumen 704. Where mechanical abrading of organized thrombus is desired the guide wire is removed from the catheter lumen 704 and the catheter assembly 500 is thereafter fed through the catheter lumen 704 to the desired location within the vasculature. In one example the catheter assembly 500 is deployed from the distal opening 716 in the stored configuration such as the stored configuration shown in FIG. 5A. In another example the catheter assembly 500 in the stored configuration is fed through the organized thrombus and thereafter deployed into the configuration shown in FIG. 7 on a side of the thrombus opposed to the thrombectomy catheter 700. In yet another example, the catheter assembly 500 including the brushes 508, 510 is fed into the organized thrombus and the brushes are thereafter deployed within the organized thrombus. In still yet another example the catheter assembly 500 is delivered proximally relative to the organized thrombus and the brushes 508, 510 are thereafter expanded proximally relative to the organized thrombus.

After delivery of the catheter assembly 500 to the desired location within the vasculature the brushes 508, 510 are deployed into the configuration shown in FIG. 7. Thereafter the brushes 508, 510 are reciprocated through movement of the catheter assembly 500 relative to the thrombectomy catheter 700 (shown with the backward and forward arrows in FIG. 7). The reciprocating movement engages the brushes 508, 510 with thrombus and abrades the thrombus along the vessel wall thereby freeing the organized thrombus for evacuation with the thrombectomy catheter 700. Optionally, the catheter assembly 500 is used in combination with the circular flow 718 generated through the inflow and outflow orifices 712, 714 of the thrombectomy catheter 700. Stated another way, the circular flow 718 and brushes 508, 510 of the catheter assembly 500 cooperate to mechanically abrade and hydrodynamically impinge organized thrombus within the vessel. By having the circular flow 718 and brushes 508, 510 work together dual thrombectomy techniques are provided at a single location of thrombus. Removal of thrombus at the desired location within the vasculature is thereby enhanced through the dual functions of mechanical abrasion and hydrodynamic impingement. Thrombus that is removed from the vessel wall is thereafter drawn into the thrombectomy catheter 700 through the inflow orifice 712 by the circular flow 718 and exhausted from the thrombectomy catheter 700 through the catheter lumen 704. Optionally, the catheter assembly 500 is operated at the same time as the thrombectomy catheter 700 or their operation is staggered. For instance, the catheter assembly 500 is operated to abrade thrombus and the thrombectomy catheter is operated thereafter to macerate and aspirate the loose thrombus.

An additional benefit of the combination of the thrombectomy catheter 700 with the catheter assembly 500 includes the centering function of the catheter assembly 500. As previously described, the proximal and distal brushes 508, 510 in the deployed configuration center the catheter assembly 500 within the local area of the vessel. In a similar manner the catheter assembly 500 in the deployed configuration shown in FIG. 7 correspondingly centers the thrombectomy catheter 700 such as the thrombectomy catheter distal portion 702 having the inflow and outflow orifices 712, 714 in the corresponding portion of the vasculature. Centering of the thrombectomy catheter 700 substantially prevents the positioning of the inflow and outflow orifices 712, 714 adjacent to the vessel wall and substantially prevents the suction of the vessel wall into the inflow orifice 712. Further, centering of the thrombectomy catheter 700 within the vessel facilitates the rotation of the thrombectomy catheter 700 including the inflow and outflow orifices 712, 714 and the corresponding circular flow 718 around the entire perimeter of the vessel. Stated another way, the thrombectomy catheter 700 is able to easily rotate through the entire perimeter and thereby engage the circular flow 718 with any organized thrombus at any radial position within the vessel.

Optionally, after delivery to a desired location within the vasculature a balloon, such as an occlusive balloon mounted on one or more of the thrombectomy catheter 700 or the catheter assembly 500, is deployed to occlude the vessel. One or more of the catheter assembly 500 and the thrombectomy catheter 700 are then operated and the balloon substantially prevents the flow of loose thrombus away from the thrombectomy location. In another example, the assembly includes two or more occluding balloons. At least one of the balloons is mounted on the atraumatic tip 111 of the catheter assembly 500. Another balloon is mounted proximally relative to one or more of the brushes 508, 510 and the inflow and outflow orifices 712, 714 (e.g., on one or the other of the catheter assembly 500 and the catheter 700). The balloons cooperate to isolate the thrombectomy operation of the catheter 700 and the catheter assembly 500 and contain loose thrombus therebetween until removed through operation of the catheter 700.

In still another example, the catheter assembly 500 including the one or more brushes 508, 510 are incorporated as part of the catheter 700. For instance, the brushes 508, 510 are positioned proximally relative to the inflow and outflow orifices 712, 714. The inner mandrel 108 extends through the catheter lumen 704 and is otherwise engaged to one of the shoulders as previously described herein. Optionally, the one or more brushes 508, 510 are positioned over the inflow and outflow orifices 712, 714 with the shoulders slidably positioned over the catheter distal portion 702.

Optionally, the catheter assembly 500 including the one or more brushes 508, 510 are used in combination with other treatment devices including, but not limited to, drug coated balloons, drug dispensing catheters (e.g., lytics, anti-coagulants and the like). In still another example, the catheter assembly 500 is positioned within a drug coated balloon and deployment of one or more of the brushes 508, 510 similarly deploys the drug coated balloon.

Referring now to FIGS. 8A, B, one example of the outer sheath 106 and inner mandrel 108 are shown. In the example shown in FIGS. 8A, B an example of a feature configured for retaining the inner mandrel 108 relative to the outer sheath 106 is provided. The inner mandrel 108 includes for instance a mandrel coin at one or more locations along the inner mandrel 108 (e.g., at a proximal, intermediate or distal end of the mandrel). For instance, the mandrel coin 800 and the associated portion of the outer sheath 106 engageable with the mandrel coin 800 are positioned in one or more locations within vessel of a subject or outside of the body of the subject. The outer sheath 106 further includes a sheath inner surface 802. The mandrel coin 800 and the sheath inner surface 802 are sized and shaped to ensure mechanical engagement between the inner mandrel 108 and the outer sheath 106 and retention of the inner mandrel at one or more positions relative to the outer sheath. For instance, in one example the mandrel coin 800 is positioned along the inner mandrel 108 to ensure the mandrel coin 800 engages with the sheath inner surface 802 where the deployable scrubber assembly 104, 504 is in the deployed configuration with the bristles 112 extended in the brush shapes described herein. Stated another way, the mandrel coin 800 only engages with the sheath inner surface 802 after the plurality of deformable struts 110 are formed into the bristles 112 shown in FIGS. 1B and 5B. When storage of the bristles 112 is desired (e.g., transition to the stored configuration) the mandrel coin 800 is moved out of engagement with the sheath inner surface 802 and the inner mandrel 108 is moved relative to the outer sheath to move the corresponding shoulders apart.

In yet another example, the mandrel coin 800 is positioned within the outer sheath 106 and thereby engaged with the sheath inner surface 802 throughout movement of the inner mandrel 108 relative to the outer sheath 106. The engagement of the mandrel coin 800 with the sheath inner surface 802 throughout movement of the inner mandrel 108 substantially ensures movement of the inner mandrel 108 including corresponding deployment and storage of the plurality of deformable struts 110 is performed with mechanical engagement therebetween. That is to say, the mandrel coin 800 and the sheath inner surface 802 provide a reliable engagement throughout movement of the inner mandrel to ensure that the plurality of deformable struts are retained in whatever position the user desires whether that position is the stored configuration shown for instance in FIG. 1A, the deployed configuration shown in FIG. 1B or an intermediate configuration therebetween. Stated another way, the user overcomes the frictional engagement between the mandrel coin 800 and the sheath inner surface 802 and slides the inner mandrel 108 relative to the outer sheath 106 to move a deployable scrubber assembly 104, 504 (see FIGS. 1A, B and 5A, B) between the deployed and stored configurations.

In another option, the mandrel 108 includes a ground taper (in contrast to a coin) that provides tapered interference engagement with the outer sheath 106 and ensures the inner mandrel 108 is securely engaged with and held in position relative to the sheath. In still another option, the mandrel 108 is ground or formed with a stepped interface (i.e., a portion with a first diameter and a second portion with a second diameter greater than the first). The stepped interface provides selective interfering engagement between the inner mandrel 108 and the sheath inner surface (802 shown in FIGS. 8A, B) according to movement of the inner mandrel relative to the sheath, for instance where the inner mandrel 108 is pulled proximally relative to the sheath 106.

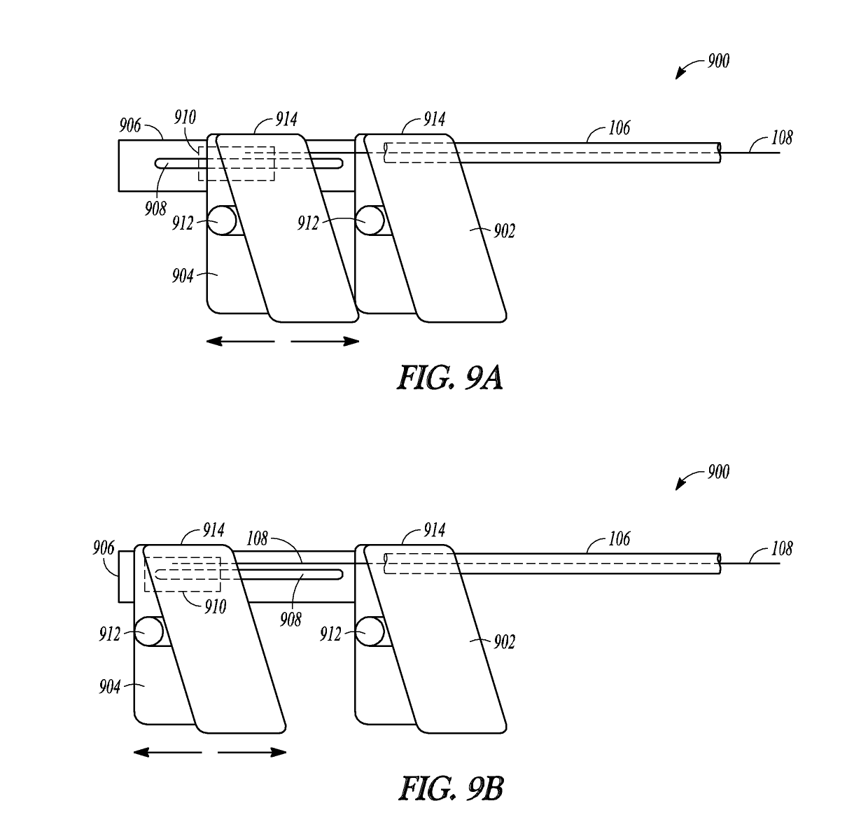

Referring now to FIGS. 9A and 9B, one example of a gripping assembly 900 is shown. The gripping assembly 900 facilitates grasping and manipulation of the inner mandrel 108 and the outer sheath 106. As shown in FIG. 9A, the gripping assembly 900 includes a distal grip 902 movably coupled with a proximal grip 904. A rail 906 extends from the distal grip 902 proximally toward the proximal grip 904. The rail 906 includes a slot 908 sized and shaped to receive a portion of a slide 910 extending through the slot and coupled along the rail 906. The slide 910 is further coupled with the proximal grip 904 to allow sliding movement of the proximal grip 904 along the rail 906. The distal and proximal grips 902, 904 further include biasing elements 912, such as hinges including torsion springs, configured to grip jaws 914 around corresponding parts of the catheter assembly 100 (or 500). For instance, the distal grip 902 includes jaws 914 sized and shaped to grip the outer sheath 106 while the proximal grip 904 includes jaws 914 configured to grip the inner mandrel 108.

The proximal grip 904 is shown in two positions in FIGS. 9A and 9B. The position shown in FIG. 9A corresponds to a stored configuration for the deployable scrubber assemblies 104, 504 (see FIGS. 1A and 5A). For instance, the inner mandrel 108 is positioned proximate relative to the outer sheath 106 thereby facilitating the stored configuration shown in FIGS. 1A, 5A. Referring now to FIG. 9B, the proximal grip 904 is shown proximally moved relative to the distal grip 902 along the rail 906. Because the jaws 914 continue to grip the inner mandrel 108 and outer sheath 106 movement of the proximal grip 904 moves the inner mandrel 108 relative to the outer sheath 106 and thereby correspondingly deploys the deployable scrubber assembly 104, 504. The distal and proximal grips 902, 904 provide easy to use and easy to hold features for the user to grasp the inner mandrel 108 and the outer sheath 106 during operation of the catheter assembly 100, 500. In another example, where the slide 910 is frictionally engaged with the surfaces of the rail 906 friction therebetween retains the inner mandrel 108 at a desired position relative to the outer sheath 106. The user is thereby able to reliably and confidently move the inner mandrel 108 relative to the outer sheath 106 and correspondingly position the deployable scrubber assembly 104, 504 in a desired configuration such as the deployed configuration (see FIGS. 1B, 5B).

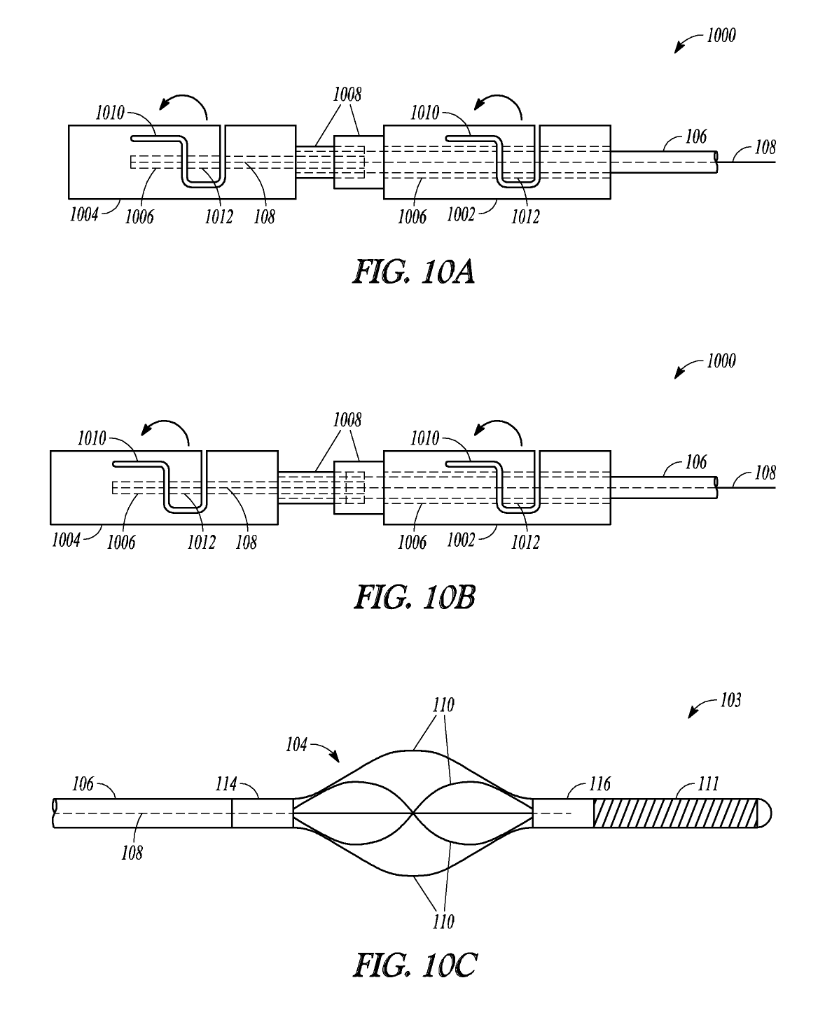

FIGS. 10A and 10B show another example of a gripping assembly 1000. Referring first to FIG. 10A, the gripping assembly 1000 includes a distal grip 1002 movably coupled with a proximal grip 1004 by telescoping tubes 1008 coupled therebetween. As described below the distal and proximal grips 1002, 1004 cooperate to permit the sliding movement of the inner mandrel 108 relative to the outer sheath 106. Further, the telescoping tubes 1008 extend around portions of the inner mandrel 108 and the outer sheath 106 and provide lateral support to one or more of the inner mandrel and the outer sheath during movement of the proximal grip relative to the distal grip to actuate a deployable scrubber assembly, such as deployable scrubber assemblies 104, 504 shown in FIGS. 1A, 5A, respectively.

Referring first to the distal grip 1002 (the proximal grip is similar in many regards to the distal grip), the grip 1002 includes a grip channel 1006 extending through the distal grip 1002 to the telescoping tube 1008 associated with the distal grip. The grip channel 1006 is sized and shaped to receive the outer sheath 106 including the inner mandrel 108 therein. The distal grip 1002 further includes a biased arm 1010 extending from the distal grip 1002. The biased arm 1010 includes an arm passage 1012 in line with the grip channel 1006. The biased arm 1010 has a bias that predisposes the biased arm 1010 into a deflected state away from the remainder of the distal grip 1002. Stated another way the biased arm 1010 biases the arm passage 1012 out of alignment with the grip channel 1006. In a similar manner, the proximal grip 1004 includes a corresponding arm passage 1012 aligned with a grip channel 1006 extending through the proximal grip 1004. The biased arm 1010 of the proximal grip 1004 biases the arm and the associated arm passage 1012 out of alignment with the corresponding grip channel 1006 in the proximal grip.

The gripping assembly 1000 is installed along the outer sheath 106 and inner mandrel 108 as shown in FIG. 10A. That is to say, the outer sheath 106 is fed through the grip channel 1006 and arm passage 1012 of the distal grip 1002 while the inner mandrel extending through the outer sheath 106 is fed by itself through the telescoping tube 1008 associated with the proximal grip 1004 and then into the grip channel 1006 and arm passage 1012 of the proximal grip. As the outer sheath 106 and inner mandrel 108 are threaded into the respective distal and proximal grips 1002, 1004 the biased arms 1010 are released and deflect outwardly away from the remainder of the associated distal and proximal grips 1002, 1004. Deflection of the biased arms 1010 tightly engages the surfaces defining the arm passage 1012 and grip channel 1006 in a shearing action with each of the respective inner mandrel 108 and outer sheath 106 and thereby tightly grasps the associated mandrel and outer sheath in the respective distal and proximal grips 1002, 1004.

Referring again to FIGS. 10A and 10B, in operation after the outer sheath 106 and inner mandrel 108 are coupled with the gripping assembly 1000 the user grasps the distal and proximal grips 1002, 1004 and moves the proximal grip 1004, for instance relative to the distal grip 1002, to correspondingly slide the inner mandrel 108 relative to the outer sheath 106. As previously described above, movement of the inner mandrel 108 relative to the outer sheath 106 correspondingly deploys or stores the deployable scrubber assembly (e.g., 104, 504). The telescoping tubes 1008 of the gripping assembly 1000 engage around the inner mandrel 108 to provide lateral support to at least the inner mandrel 108 during movement of the proximal grip 1004 relative to the distal grip 1002. That is to say, the user is able to move the proximal grip 1004 backward and forward relative to the distal grip 1002 with corresponding compressive loading of the inner mandrel 108, and the telescoping tubes 1008 extend around and engage with the inner mandrel to provide lateral support and substantially prevent buckling and kinking of the inner mandrel 108.

In another example, a single grip, such as the proximal grip 1004, is coupled with the inner mandrel 108. The user grasps the proximal grip 1004 and moves the grip and the mandrel 108 relative to the outer sheath 106 to deploy and store one or more of the scrubber assemblies (e.g., 104, 504). In one example, the user grasps the outer sheath 106 with an opposed hand, a clamp or the like.

Referring now to FIG. 10C the frictional engagement between the inner mandrel 108 and the outer sheath 106 in one example allows for the selective deployment of the plurality of deformable struts 110. For instance if the user desires to deploy the plurality of deformable struts 110 into a cage configuration as shown in FIG. 10C movement of the inner mandrel 108 relative to the outer sheath 106 is performed so that the distal shoulder 116 is moved proximally toward the proximal shoulder 114 without engagement therebetween. For instance, where the user desires to use the plurality of deformable struts 110 as an embolic cage the inner mandrel 108 is withdrawn relative to the outer sheath 106 and the distal shoulder 116 is correspondingly pulled toward but not engaged with the proximal shoulder of 114. In this configuration the deployable scrubber assembly 104 including the plurality of deformable struts 110 is used as an embolic cage or filter. When used in combination with the gripping assembly 1000 or the gripping assembly 900 described previously the user is able to easily grasp the inner mandrel 108 and outer sheath 106 to quickly and reliably actuate the deployable scrubber assembly 104 by grasping the grips of the respective gripping assembly 900, 1000 and move the grips relative to each other. The gripping assemblies 900, 1000 facilitate the accurate and reliable deployment of the deformable struts into any desired configuration (e.g., stored, fully deployed into brushes and any of a number of intermediate configurations). Alternatively the user operates the inner mandrel 108 relative to the outer sheath 106 by grasping the inner mandrel 108 and moving the inner mandrel relative to the outer sheath 106 without the gripping assemblies.

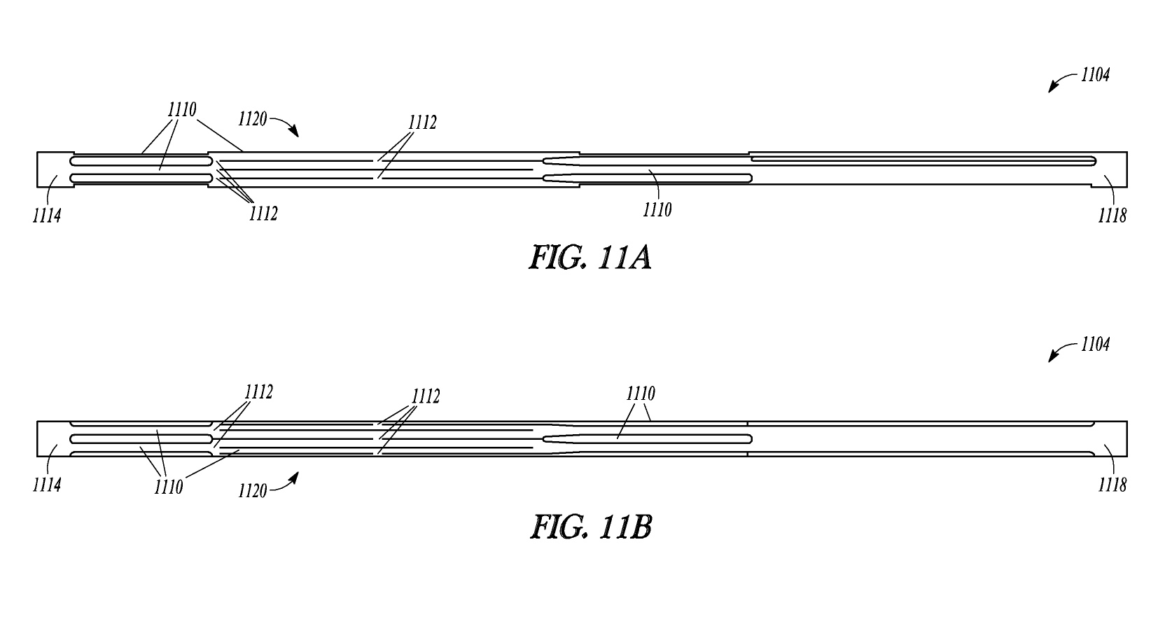

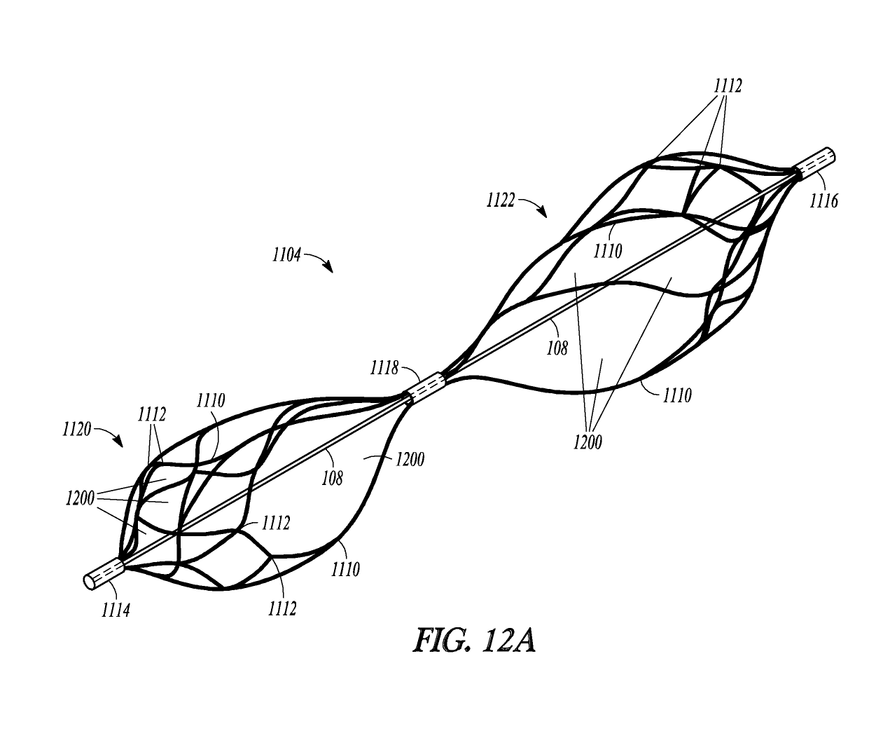

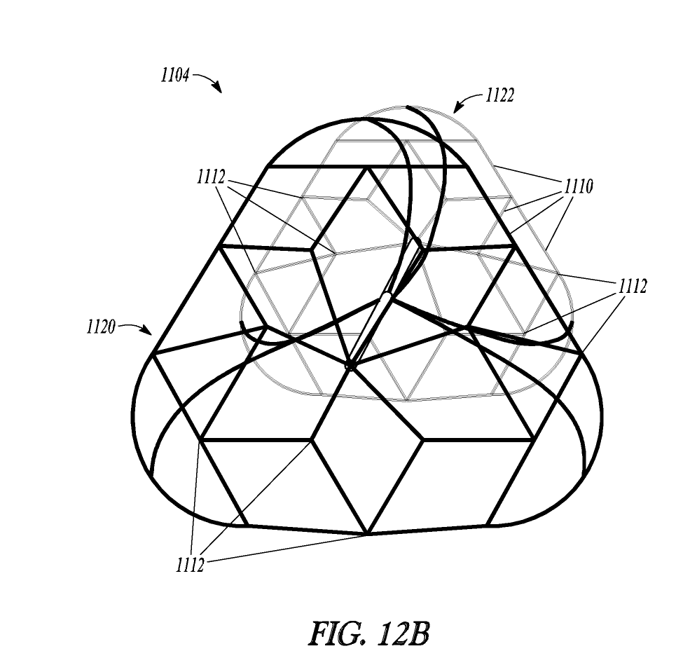

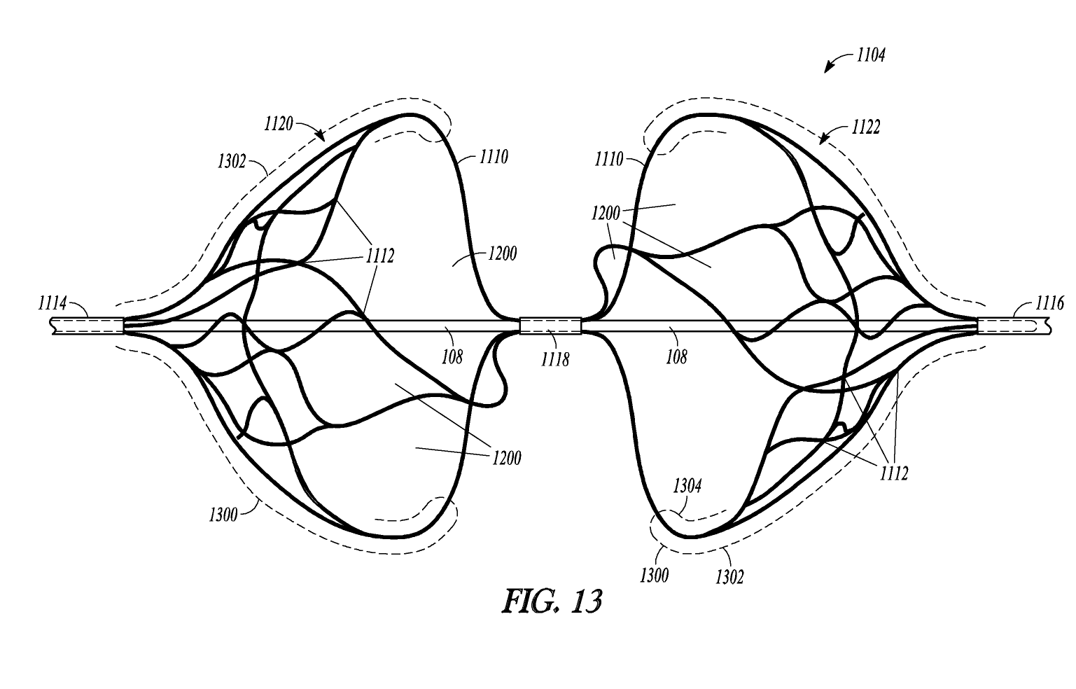

FIGS. 11A, B show another example of a deployable scrubber assembly 1104. FIG. 11B is rotated approximately 45 degrees relative to the view provided in FIG. 11A. As with the previous examples, the deployable scrubber assembly 1104 includes one or more brushes 1120, 1122 (FIGS. 11A, B show the proximal brush 1120, while later FIGS. 12A, B and the like show both brushes 1120, 1122) including struts 1110 and proximal, distal and intermediate shoulders 1114, 1116, 1118 at the ends of each of the proximal and distal brushes. The intermediate and distal shoulders 1118 and 1116 as well as the distal brush 1122 are shown in FIGS. 12A, B and the Figures following thereafter. The deployable scrubber assembly 1104 is coupled, for instance, between the inner mandrel 108 and the outer sheath 106 of a catheter assembly, such as the catheter assembly 100 shown in FIG. 1A. In the example shown in FIGS. 11A, B, the struts 1110 of the deployable scrubber assembly 1104 include a plurality of nodes 1112 connecting one or more of the struts 1110 to each other between the proximal and intermediate shoulders 1114, 1118. As previously described, the provision of nodes 1112 along the struts 1110 changes the manner in which the struts 1110 deploy as the deployable scrubber assembly 1104 is moved from the stored configuration shown in FIGS. 11A, B into the partially and fully deployed configurations described herein. That is to say, the nodes 1112 are provided at specified locations along the struts 1110 and in various configurations along the struts to facilitate expansion and opening of the proximal and distal brushes 1120, 1122 into desired configurations including but not limited to two-dimensional and three-dimensional configurations. In some examples, as described below, the two-dimensional and three-dimensional configurations include baskets, toroids, cylinders, ellipses and the like. The nodes 1112 provide increased numbers of cells within the deployable scrubber assembly and alter the brushing characteristics of the scrubber assembly. For example, the nodes 1112 provide more compact cells and concentrate the cutting surface of the brushes into a tighter space.

FIGS. 12A and 12B show the deployable scrubber assembly 1104 in first partially deployed configurations where the proximal and distal brushes 1120, 1122 are partially deployed away from the inner mandrel 108. As previously described, the proximal and distal brushes 1120, 1122 are coupled with the inner mandrel 108 as well as the outer sheath 106 as shown in FIGS. 2A and 2B with the deployable scrubber assembly 104. Movement of the inner mandrel 108, for instance, proximally relative to the outer sheath 106 pulls the distal shoulder 1116 toward the intermediate shoulder 1118 and the intermediate shoulder 1118 is correspondingly pulled toward the proximal shoulder 1114. The struts 1110 (including the nodes 1112 extending therebetween) are expanded into the partially deployed configuration shown in FIG. 12A. As shown in FIG. 12A, the nodes 1112 bond the struts 1110 at various locations between the proximal and intermediate shoulders 1114, 1118 as well as the intermediate and distal shoulders 1118, 1116. Provision of the nodes 1112 increases the number of cells 1200 between the struts 1110.

As shown in FIG. 12A provision of the nodes 1112, for instance, near the proximal shoulder 1114 and the distal shoulder 1116 concentrates the cells 1200 into a tightly packed cluster adjacent to the proximal and distal shoulders 1114, 1116. In contrast, there are fewer nodes 1112 (and cells 1200) adjacent to the intermediate shoulder 1118. The provision of a greater number of nodes 1112 and cells 1200 near the proximal and distal shoulders 1114, 1116 facilitates the use of the deployable scrubber assembly 1104 in this partially deployed configuration to retain and hold thrombus between the proximal and distal brushes 1120, 1122 as the deployable scrubber assembly 1104 is reciprocated to remove thrombus from the vessel. Stated another way, the nodes 1112 and the corresponding cells 1200 having smaller openings relative to the cells adjacent to the intermediate shoulder 1118 provide a tightly packed matrix that collects loose thrombus between the proximal shoulder 1114 and the distal shoulder 1116 during a thrombectomy procedure. Optionally, each of the proximal and distal brushes 1120, 1122 includes differing configurations of nodes 1112 and struts 1110 to facilitate staggered deployment of one of the brushes 1120, 1122 relative to the other brush. For instance, the proximal brush 1120 includes one or more of wider struts 1110 or a greater number of nodes 1122 relative to the distal brush 1122, and the distal brush 1122 (with a looser arrangement of struts 1110) is thereby easily biased into the deployed configuration before the proximal brush 1120.

Referring now to FIG. 12B, the plurality of nodes 1112 are shown in an end view of the deployable scrubber assembly 1104. As shown, the nodes 1112 near the proximal and distal shoulders 1114, 1116 (see FIG. 12A) provide a tightly packed matrix of cells 1200. As previously described in other examples, providing the plurality of nodes 1112 provides additional cutting features (e.g., intersections between struts) for engagement with thrombus within a vessel. By deploying the proximal and distal brushes 1120, 1122 with the plurality of nodes 1112 and corresponding cells 1200 the abrading of thrombus from within the vessel is enhanced at the junctures created with the nodes 1112.

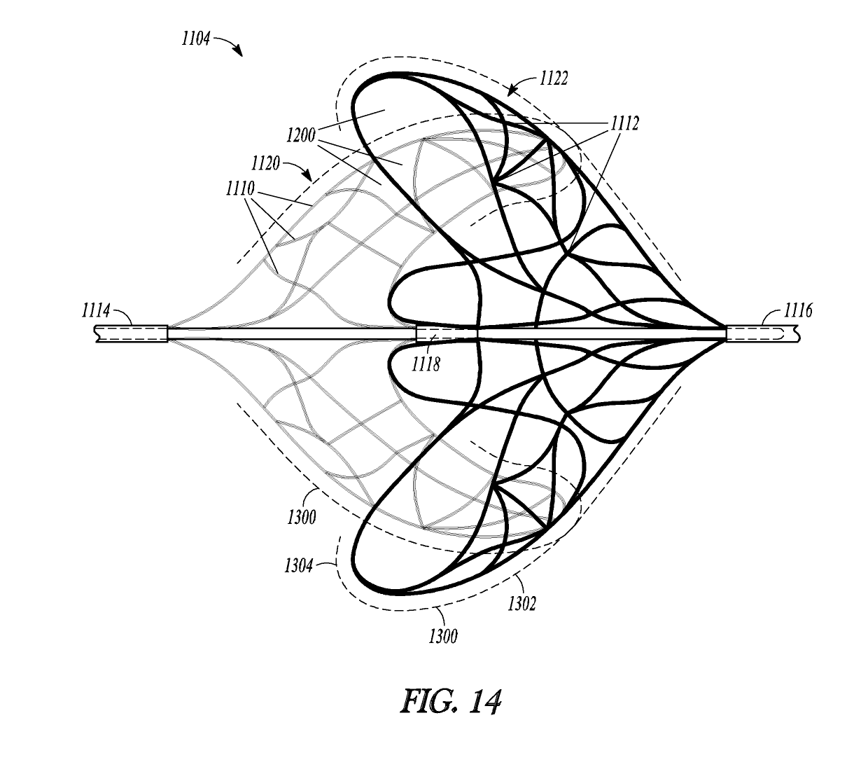

FIG. 13 shows the deployable scrubber assembly 1104 in a first intermediate deployed configuration with the distal shoulder 1116 pulled closer to the proximal shoulder 1114 relative to the orientation shown in FIGS. 12A and 12B. As shown, the intermediate shoulder 1118 between the proximal and distal shoulders 1114, 1116 is positioned closer to the shoulders 1114, 1116 and the struts 1110 extending therebetween are correspondingly further deflected into a deployed configuration. As previously described, the nodes 1112 are more numerous adjacent to the proximal and distal ends 1114, 1116 of the deployable scrubber assembly 1104. Correspondingly, as shown in FIG. 3, with continued deflection of the deformable struts 1110 into the configuration shown the structure of the proximal and distal brushes 1120, 1122 is more tightly constrained adjacent to the proximal and distal shoulders 1114, 1116 than it is adjacent to the intermediate shoulder 1118. Stated another way, as shown in FIG. 13, the deformable struts 1110 adjacent to the intermediate shoulder 1118 are free to expand away from the inner mandrel 108. As shown in FIG. 13, the struts adjacent to the intermediate shoulder 1118 extend orthogonally away from the inner mandrel 108.

As shown in FIG. 13 the proximal and distal brushes 1120, 1122 form everted cones 1300 (see the dashed lines). The everted cones 1300 include a cone exterior 1302 comprised mainly of the portion of the proximal and distal brushes 1120, 1122 having a higher density of nodes 1112 relative to those portions of brushes having less frequent nodes, for instance, near the intermediate shoulder 1118. Similarly, the cone interiors 1304 of each of the proximal and distal brushes 1120, 1122 include those portions of the brushes having struts 1120 that are less frequently bonded together by nodes 1112. Stated another way, the portions of the brushes 1120, 1122 including a higher frequency of nodes 1112 are more structurally robust and resistant to deflection caused by movement of the proximal, intermediate and distal shoulders 1114, 1118, 1116, respectively. Conversely, the deformable struts 1110 adjacent to the intermediate shoulder 1118 are less constrained because of the less frequent nodes 1112 and are thereby able to easily deflect when under stress caused by the movement of the inner mandrel 108 relative to the outer sheath 106.

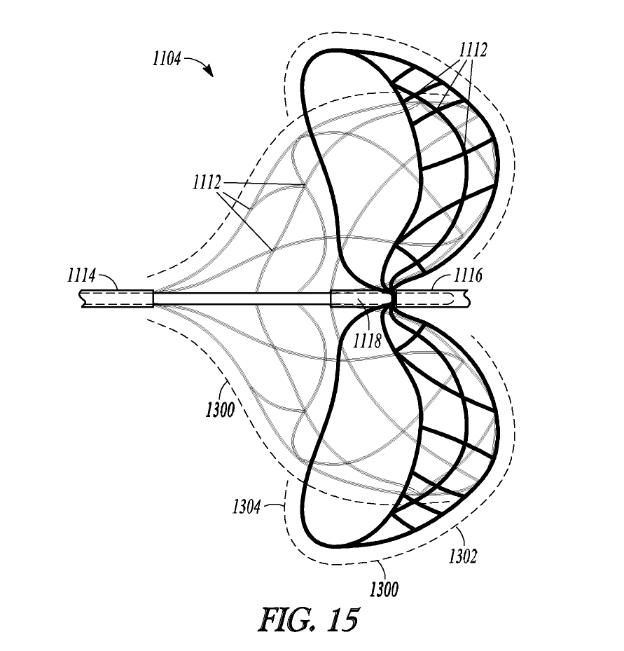

By providing the nodes 112 and the corresponding cells 1200 at various locations along the struts 1110 the shape of the proximal and distal brushes 1120, 1122 during deployment and at full deployment is tuned according to the needs of the particular procedure and desired capabilities for the deployable scrubber assembly 1104. For instance, with a relatively large plurality of nodes 1112 a tight and structurally rigid configuration of the brushes 1120, 1122 is provided for enhanced cutting and removal of organized thrombus. In another example, the plurality of deformable struts 1110 include fewer nodes 1112 than in the previously discussed example, and expand away from the inner mandrel easily. This configuration assists in providing an embolic filter that extends across a vessel and facilitates capture of removed thrombus. When used in cooperation with a thrombus removal device, including but not limited to, a thrombectomy catheter 700 (see FIG. 7), captured thrombus is aspirated from within the brushes 1120, 1122 (nested as shown in FIGS. 14 and 15 or spaced apart in FIG. 13). Optionally, the brushes 1120, 1122 are transitioned to the stored configuration shown in FIGS. 11A, B and any thrombus captured within the brushes 1120, 1122 is pulled into the thrombectomy catheter 700. Thrombus released by the transition to the stored configuration is pulled into the circular flow 718 of the thrombectomy catheter 700 and eventually withdrawn from the vessel through the inflow orifice 712. In still other examples, a single brush (like brushes 1120, 1122) includes regions with varying numbers of nodes to provide regions for cutting and removal of thrombus and capture of thrombus.

FIG. 14 shows another example of the deployable scrubber assembly 1104 in a second intermediate deployed configuration after the proximal and distal shoulders 1114, 1116 are moved closer relative to the configuration shown in FIG. 13. As previously described, with sufficient movement of the inner mandrel 108 relative to the outer sheath 106 (see FIGS. 1A, B) the proximal and distal brushes 1120, 112 provide everted cones 1300 with cone exteriors 1302 and cone interiors 1304 that are generated by the continued deflection of the deformable struts 1110 according to the placement of a plurality of nodes 1112 along the deformable struts 1110. Stated another way, with continued deflection the struts 1110 adjacent to the intermediate shoulder 1118 evert and form the cone interiors 1304.

As shown in FIG. 14, continued deflection of the plurality of deformable struts 1110 of the deployable scrubber assembly 1104 nests the proximal brush 1120 including its everted cone 1300 within the everted cone 1300 of the distal brush 1122 (see the dashed lines showing the nesting of the proximal everted cone 1300 within the distal everted cone 1300). The deployable scrubber assembly 1104 in the second intermediate deployed configuration is formed into a bulb shape having one or more petals or bristles corresponding to the deflected deformable struts 1110 of the outer everted cone 1300 of the distal brush 1122. By nesting the proximal brush 1120 within the distal brush 1122 a structural support is provided beneath the distal everted cone 1300 to maintain the cone exterior 1302 in the deployed configuration if and when the deployable scrubber assembly 1104 is reciprocated to remove thrombus from the vasculature. Stated another way, the deployable scrubber assembly 1104 includes a structural support provided by the proximal brush 1120 that engages against the cone interior 1304 of the distal brush 1122 and ensures the distal brush 1122 remains in the deployed configuration shown in FIG. 14 during reciprocating movement (proximal and distal movement) for removal of thrombus along the vessel wall.

In one example, the proximal brush 1120 (with a first everted cone 1300) is nested within the distal brush 1122 (having the second everted cone 1300) according to the configuration of struts 1110 and nodes 1112. Stated another way, the nodes 1112 are located along the struts 1110 (e.g., with greater frequency, spaced differently and the like) to bias the proximal brush 1120 into a nesting engagement within the distal brush 1122 as shown in FIG. 14. Optionally, the struts 1110 in the proximal brush include differing widths or a configuration of struts (e.g., different spiral cut pattern) that biases the proximal brush into nesting engagement within the distal brush 1122. In still another example, the proximal brush 1120 is sized smaller than the distal brush 1122 to facilitate the nesting shown in FIG. 14. Optionally, one or both of the width of the struts 1110 and positioning of nodes 1112 are specified to ensure nesting of the proximal or distal brush 1120, 1122 within the other of the distal or proximal brush. Alternatively, the distal brush 1122 includes a first material configured to expand into a larger configuration than the proximal brush 1120. The proximal brush 1120 includes a second material (e.g., a stiffer material) that facilitates deployment to a smaller configuration capable of nesting within the distal brush 1122.

FIG. 15 shows the deployable scrubber assembly 1104 in a near fully deployed configuration where at least the distal shoulder 1116 is engaged with the intermediate shoulder 1118. As previously described, engagement of one or more of the shoulders 1116, 1118 (in another example, the proximal shoulder 1114) is provided when the inner mandrel 108 is moved proximally relative to the outer sheath 106. As previously described, the engagement of the shoulders 1114, 1118, 1116 (for instance, through the bases of the struts 1110) provides a rigid structural base to the proximal and distal brushes 1120, 1122 and facilitates enhanced removal of thrombus from within the vessel. That is to say, the engagement of the shoulders 1114, 1116, 1118 provides a rigid structural base to the deployed proximal and distal brushes 1120, 1122 and substantially prevents the collapse of the brushes when moved reciprocally within a vessel to engage with and abrade thrombus from the vessel walls.

As previously described, the nesting of the proximal brush 1120 within the distal brush 1122, for instance, by moving the proximal everted cone 1300 into the distal everted cone 1300 engages the cone exterior 1302 in the proximal cone with the cone interior 1304 of the distal cone 1300. The structural support provided by the engagement of the proximal brush 1120 with the distal brush 1122 provides an enhanced structural support to the distal brush 1122 and further cooperates with the engagement of the shoulders 1116, 1118 to provide an even stronger structural base for the distal brush 1120 during reciprocation of the deployable scrubber assembly 1104 for thrombectomy. Continued proximal movement of the inner mandrel 108 further deflects the deformable struts 1110 until the proximal and intermediate shoulders 1114, 1118 engage. The proximal everted cone 1300 of the proximal brush 1120 is fully nested within the distal everted cone 1300 and cooperates with the engaged shoulders 1114, 1118 to further enhance the structural support for the distal brush 1122 (distal everted cone 1300).

As previously described the provision of the plurality of nodes 1112 at specified locations along the deformable struts 1110 facilitates the deflection of struts into the shapes shown in FIGS. 11 through 15. In a similar manner, the struts 1110 including a plurality of nodes positioned along the struts 1110 in other examples facilitate the deployment of the struts into one or more two-dimensional or three-dimensional configurations according to the needs of the procedure and desired configuration for the deployable scrubber assembly. Deployable scrubbers assemblies are thereby configured with one or more brushes with differing node and cell configurations to provide one or more brushes 1120, 1122 that deploy into toroidal shapes, triangular shapes, ovular shapes, ellipses and the like. For instance, where a robust configuration needing a plurality of cells 1200 formed with a plurality of nodes 1112 is needed the nodes 1112 are tightly packed along the deformable struts 1110. Upon deployment the nodes 1112 ensure that a plurality of cutting surfaces are generated for engagement and remove of organized thrombus within a vessel. Similarly, the nodes 1112 ensure the deformable struts 1110 deploy into a desired configuration having sufficient structural rigidity to ensure the deformable struts 1110 (for instance, bristles) are structurally supported during reciprocating movement of the deployable scrubber assembly within a vessel.

Optionally, the deployable scrubber assemblies described herein including one or more of the brushes actuated with the inner mandrel 108 and the outer sheath 106 are selectively deployed according to one or more of the size of a vessel, the treatment performed and the like. For instance, the brushes of the deployable scrubber assemblies, such as brushes 1120, 1122 are deployed until the brushes have an effective diameter slightly larger than the vessel diameter. The brushes 1120, 1122 are thereby able to abrade and remove thrombus without harming the vessel wall. In one example, the inner mandrel 108 and the outer sheath 106 include gripping mechanisms (described herein) that facilitate the selective deployment of the scrubbing assemblies with desired effective diameters and retention of the assemblies at the desired diameters.

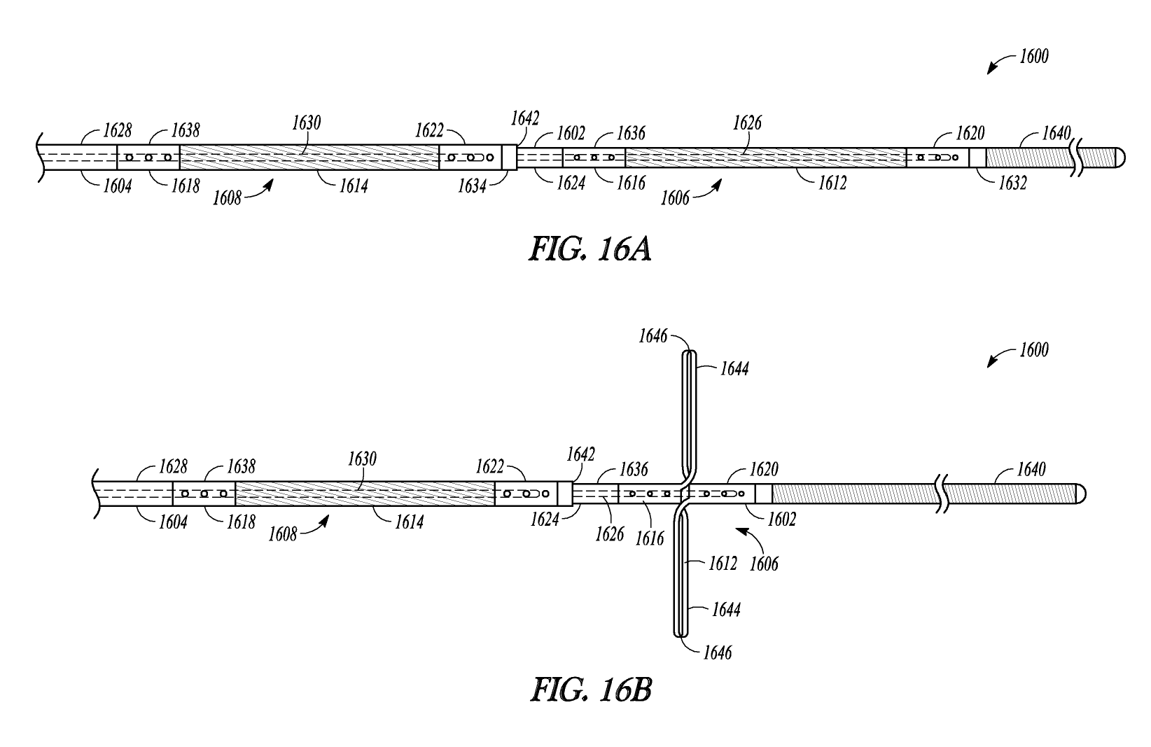

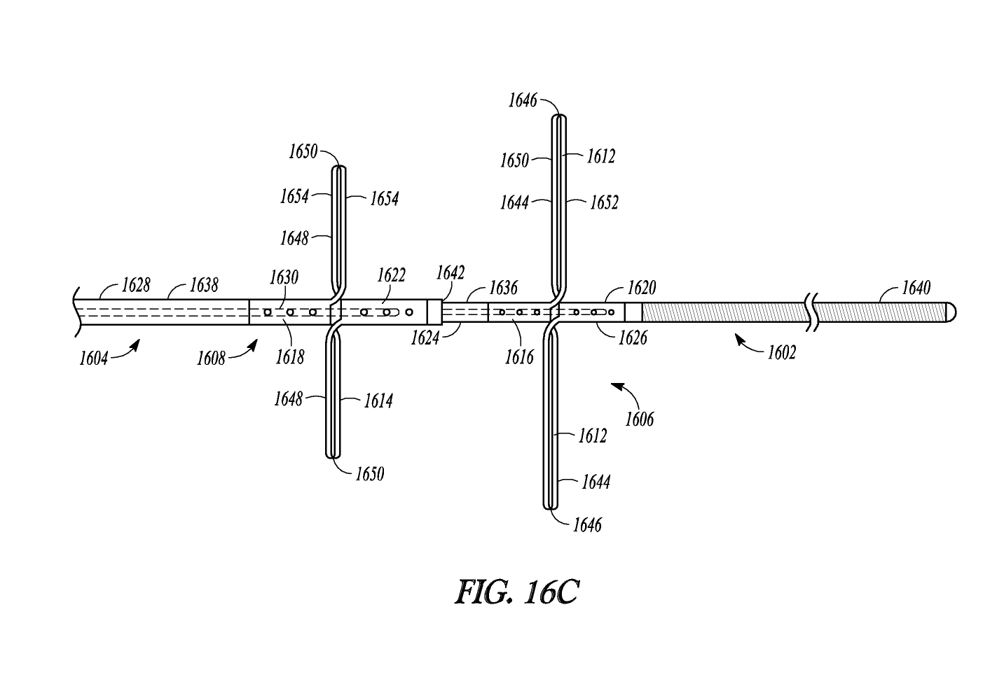

FIG. 16A shows another example of a catheter assembly 1600. The catheter assembly 1600 includes a first scrubbing catheter 1602 slidably received within a second scrubbing catheter 1604. Each of the first and second scrubbing catheters 1602, 1604 include respective first and second scrubbing assemblies 1606, 1608. The scrubbing assemblies 1606, 1608 are similar in at least some regards to the previously described scrubber assemblies herein. For instance, the first and second scrubber assemblies 1606, 1608 each include a plurality of deformable struts 1612, 1614 (e.g., a first plurality of deformable struts 1612, and a second plurality of deformable struts 1614). As further shown in FIG. 16A, the first plurality of deformable struts 1612 are coupled between a first proximal shoulder 1616 and a first distal shoulder 1620 of the first scrubbing catheter 1602. Similarly, the second plurality of deformable struts 1614 are coupled between a second proximal shoulder 1618 and a second distal shoulder 1622 of the second scrubbing catheter 1604.

As shown in FIG. 16A, the first scrubbing catheter 1602 is slidably received within the second scrubbing catheter 1604. For instance, the second scrubbing catheter 1604 includes a second catheter lumen 1638 sized and shaped to slidably receive the first scrubbing catheter 1602 therein. As further shown in FIG. 16A, the second scrubbing catheter 1604 includes a second catheter distal mouth 1642 sized and shaped to allow for a smooth sliding passage of the first scrubbing catheter 1602 including, for instance, the first scrubbing assembly 1606 therethrough. In one example, the first scrubbing catheter 1602 includes an atraumatic tip 1640 to facilitate delivery of the catheter assembly 1600 through the vasculature while substantially minimizing abrasions, piercing and the like by the catheter assembly.

As previously described with the other examples herein, the catheter assembly 1600 includes a plurality of scrubbing assemblies 1606, 1608 associated with each of the respective first and second scrubbing catheters 1602, 1604. As with the previous examples each of the first and second scrubbing assemblies 1606, 1608 include multiple struts 1612, 1614 coupled between the first and second proximal shoulder 1616, 1618 and the first and second distal shoulder 1620, 1622, respectively. In a similar manner to the previous embodiments, each of the first and second scrubbing catheters 1602, 1604 further include outer sheaths 1624, 1628 and corresponding inner mandrels 1626, 1630. With reference first to the first scrubbing assembly 1606, the first outer sheath 1624 is coupled for instance with the first proximal shoulder 1616 and the first inner mandrel 1626 is correspondingly coupled with the first distal shoulder 1620. Relative movement between the first outer sheath 1624 and the first inner mandrel 1626 for instance by pulling of the inner mandrel 1626 deflects the plurality of deformable strut 1612 into a deployed configuration such as the configuration shown in FIG. 16B.

In a similar manner, the second proximal shoulder 1618 and the second distal shoulder 1622 of the second scrubbing assembly 1608 are respectively coupled with the second outer sheath 1628 and the second inner mandrel 1630. In one example, the second inner mandrel 1630 is positioned within the second catheter lumen 1638 of the second scrubbing catheter 1604 (in a similar manner the first inner mandrel 1628 is positioned within the first catheter lumen 1636 as shown in FIG. 16A). Relative movement of the second inner mandrel 1630, for instance, with respect to the second outer sheath 1628 similarly deflects the plurality of deformable struts 1614 of the second scrubbing assembly 1608 into a brush configuration such as that shown in FIG. 16C.

Each of the respective first and second scrubbing assemblies 1606, 1608 is separately deployable according to the operation of the respective first and second inner mandrels 1626, 1630 and the first and second outer sheaths 1624, 1628. That is to say, each of the first and second scrubbing catheters 1602, 1604 includes its own scrubbing assembly 1606, 1608 that are separately deployable according to the needs of the physician during a thrombectomy procedure. For instance, where the physician desires only to deploy one of the scrubbing assemblies 1606, 1608 the physician need only operate the corresponding mandrel and outer sheath of that respective scrubbing assembly to thereby deploy the one set of the deformable struts 1612, 1614 into the deployed configuration. Conversely, where deployment of one or both of the scrubbing assemblies 1606, 1608 is desired the physician need only operate the first and second inner mandrels 1626, 1630 of the respective scrubbing assemblies to thereby deploy the deformable strut 1612, 1614 into the bristle configuration shown herein.\

Additionally, the physician may deploy either the first or the second scrubbing assembly 1606, 1608 in whatever order desired according to the needs of the procedure. For instance, the physician may deploy the second scrubbing assembly 1608 first and the first scrubbing assembly 1606 second or may reverse the order to capitalize on different properties of each of the deformable struts 1612, 1614. For instance as described herein, in one example the first scrubbing assembly 1606 has a greater diameter or width when deployed relative to the second scrubbing assembly 1608 (see FIG. 16C) thereby allowing the first scrubbing catheter 1602 to center the catheter assembly 1600 within a vessel while the second scrubbing, for instance having a thicker bristle assembly with one or more sharp cutting edges, is able to easily reciprocate backward and forward over the statically positioned first scrubber catheter 1602 to thereby easily remove thrombus from the vessel.

Referring now to FIG. 16B, the catheter assembly 1600 is shown in a partially deployed configuration. The first scrubbing catheter 1602 is in a deployed configuration with the first proximal shoulder 1616 and the first distal shoulder 1620 in a close engaging configuration wherein the bases of the deformable struts 1612 are adjacent to one another and thereby provide for engagement between the first proximal and distal shoulders 1616, 1620. As previously described herein, the engagement of the struts at their bases provides for a robust structural support to the first bristles 1644 and allows for the first bristle tips 1646 to fully extend away from the catheter assembly 1600, for instance, relative to the first proximal and distal shoulders 1616, 1620. As shown, each of the bristles 1644 includes a corresponding first bristle tip 1646 extending away from the first proximal and distal shoulder 1616, 1620. As will be described further herein, in one example, the remotely extending first bristle tips 1646 allow for centering of the first scrubbing catheter 1602 within a vessel and correspondingly center the second scrubbing catheter 1604.

As previously described, in one example, the first plurality of deformable struts 1612 are deflected into the brush configuration (deployed configuration) through movement of the first inner mandrel 1626 relative to the first outer sheath 1624. For instance, the first inner mandrel 1626 is pulled relative to the first outer sheath 1624 thereby deflecting the plurality of the formable struts 1612 into the twisted bristle configuration shown in FIG. 16B. That is to say the plurality of deformable struts 1612 are deflected into the first bristles 1644 and thereby provide one or more bristles 1644 configured for sweeping back and forth movement along the vessel wall to thereby remove thrombus deposited along the vessel wall. Conversely, the first bristles 1644 are able to center the catheter assembly 1600 within the vessel to subsequently allow for reciprocation of the second scrubbing assembly 1608, for instance, with its own deflected deformable struts 1614 to conduct a thrombectomy operation wherein the second bristles of the deformable struts 1614 remove thrombus from the vessel wall while the first scrubbing assembly 1606 centers the catheter assembly 1606 therein. In yet another example, both the first and second pluralities of deformable struts 1612, 1614 are deflected into the deployed configuration and both are used simultaneously, alternately or the like to abrade and remove thrombus, for instance, with a sweeping reciprocating action of the brushes along the vessel wall.

Referring now to FIG. 16C, the catheter assembly 1600 is shown again in a fully deployed configuration with the first scrubbing assembly 1606 in a deployed configuration with the first bristles 1644 including the first bristle tips 1646 extending away from the first proximal and distal shoulders 1616, 1620. In a similar manner, the second bristles 1648 corresponding to the deflected deformable struts 1614 of the second scrubbing assembly 1608 are also deployed. In the example shown in FIG. 16C, the second bristles 1648 are fully deployed with the second proximal shoulder 1618 and second distal shoulder 1622 substantially engaged. For instance the bases of the struts 1614 are engaged with the opposing shoulders 1618, 1622 to provide a robust structural base for each of the second bristles 1648. The second bristles 1648 are in a twisted planar configuration relative to the longitudinal axis of the second scrubbing catheter 1604. As with the first scrubbing assembly 1606, the second bristles 1648 with the robust structural base previously described herein allow for a sweeping movement of the second bristles 1648, for instance, along a vessel wall to allow for the abrading of thrombus from the vessel wall through a reciprocating backward and forward movement of the second scrubbing catheter 1604 (e.g., relative to the first scrubbing catheter 1602).

The second scrubbing assembly 1608 including the second plurality of deformable struts 1614 is deflected into the deployed configuration shown in FIG. 16C in a manner similar in at least some regards to the deployment of the first scrubbing assembly 1606. For instance, the second inner mandrel 1630 is pulled relative to the second outer sheath 1628 to correspondingly move the second distal shoulder 1622 proximally relative to the second proximal shoulder 1618 and thereby deflect the second plurality of deformable struts 1614 therebetween into the second bristles 1648 shown in FIG. 16C.

In the deflected configuration the second bristles 1648 are shorter or have a smaller deployed length relative to the deployed first bristles 1644. As previously described herein, the second bristles 1648 in this configuration are able to easily reciprocate along the first scrubbing catheter 1602 such as the first outer sheath 1624 to easily allow for reciprocation of the second bristles 1648 relative to a vessel and the first scrubbing assembly 1606 statically positioned within the vessel with the first bristles 1644. That is to say, in one example, the first scrubbing assembly 1606 provides an anchor and screen for the second scrubbing assembly 1608. After positioning of the first bristles 1644 along a vessel wall the second bristles 1648 of the second scrubbing assembly 1608 are reciprocated back and forth to remove thrombus from the vessel walls.

In one example, the catheter assembly 1600 is used in combination with an aspiration catheter or a thrombectomy catheter such as the thrombectomy catheter 700 previously shown in FIG. 7. The aspiration provided by the inflow orifices 1712 as well as the distal opening 716 is configured to draw free thrombus from the vessel wall by the reciprocation of one or both of the first and second scrubbing assembly 1606, 1608 into the thrombectomy catheter 700 to thereby facilitate removal of the thrombus and clearing of the vessel. In one example, the first bristles 1644 are deflected into a configuration (see one or more of the rosette, cup and other configurations shown herein) that substantially prevents or minimizes the passage of loose thrombus distally relative to the catheter assembly 1600. Instead, the first scrubbing assembly 1606 provides a screened volume that allows the thrombectomy catheter 700 (or another aspiration catheter) to vacuum the loose thrombus therein and substantially prevent passage of thrombus outside of the treatment area.

In operation, the catheter assembly 1600 is deployed through the vasculature to a treatment location within a vessel. For instance, the first scrubbing catheter 1602 is delivered through the vessel to a location of interest, for instance, a thrombus deposit within the vessel. The second scrubbing catheter 1604, in one example, is delivered along the first scrubbing catheter 1602 into a position similar to that shown in FIG. 16A. In another example, the first and second scrubbing catheters 1602, 1604 are delivered together through the vasculature to the location of interest. In yet another example, the second scrubbing catheter 1604, for instance, with an obturator, guidewire and the like is delivered to the location of interest within the vasculature and the first scrubbing catheter 1602 is thereafter delivered down the second catheter lumen 1638 shown in FIG. 16A. Optionally, one or more markers 1632, 1634 associated with the respective catheters 1602, 1604 are used to assist with delivery of the catheters through the vasculature. The markers include, for instance, fluoroscopic markers.

After delivery of the catheter assembly 1600 to a location of interest, in one example the first scrubbing catheter 1602 is deployed. As shown, for instance in FIG. 16B, the first scrubbing assembly 1606 is deployed by deforming the plurality of deformable struts 1612 into the deployed configuration. In one example, the plurality of deformable struts 1612 are deflected into the first bristles 1644 having first bristle tips as shown in 1646. In one example, the first bristles 1644 such as the deformable struts 1614 are relatively easy to deflect relative to the deformable struts 1614 of the second scrubbing assembly 1608. For instance, the deformable struts 1612 have a narrower configuration (a narrow width between adjacent cuts) and thereby easily deflect into the first bristles 1644 shown in FIG. 16B. As will be described in further detail below, the soft deformable character of the first bristles 1644 allows for easy positioning and centering of the catheter assembly 1600 along the vessel wall while minimizing damage to the wall, for instance with a longer bristle 1644 relative to the shorter more rigid bristles 1648 of the second scrubbing assembly 1608.

As described above, deployment of the first scrubbing assembly 1606 allows for centering of the catheter assembly 1600 within the vessel. After the first scrubbing assembly 1606 is deployed the second scrubbing assembly 1608 is optionally deployed as shown in FIG. 16C. As previously described, in one example the second scrubbing assembly 1608 is deployed in a similar manner to the first scrubbing assembly 1606. For instance, the second distal shoulder 1622 is withdrawn relative to the second proximal shoulder 1618, for instance by operation of the second inner mandrel 1630. The plurality of deformable struts 1614 are correspondingly deflected into the deployed configuration shown in FIG. 16C including the second bristles 1648 with the second bristle tips 1650.

With the first and second scrubbing assembly 1606, 1608 deployed as shown in FIG. 16C the catheter assembly 1600 is then operated to remove thrombus from the location of interest within the vessel. In one example, both of the first and second scrubbing assemblies 1606, 1608 (the first and second scrubbing catheter 1602, 1604) are reciprocated within the vessel to remove thrombus from the vessel walls in a complementary fashion. In another example, the first scrubbing catheter 1602 remains substantially static with the first bristles 1644 extending away from the first proximal and distal shoulders 1616, 1620 and into an atraumatic engagement with the vessel wall. The shorter second bristles 1648 are then reciprocated by reciprocation of the second scrubbing catheter 1604 along the first scrubbing catheter 1602. For instance, the second scrubbing catheter 1604 is reciprocated along the first scrubbing catheter 1602 at the second catheter distal mouth 1642. The second catheter distal mouth 1642 provides the sliding interface (along with the outer and inner surfaces of the catheters 1602, 1604) to facilitate the reciprocal movement of the second scrubbing catheter 1604 relative to the first scrubbing catheter 1602. Additionally, with the shorter second bristle 1648 the second scrubbing assembly 1608 is easily able to reciprocate within the vessel relative to the first scrubbing catheter 1602.

In one example, the second bristles 1648 are constructed in a stiffer manner relative to the construction of the first bristles 1644. For instance, the second bristles 1648 have at least portions thereof having wider deformable struts 1614 that thereby form correspondingly more robust and stiff second bristles 1648 (compared to the first bristles 1644). In another example, the second bristles 1648 include sharper leading edges. For instance, the edges of the deformable struts 1614 are sharper relative to the edges of the deformable struts 1612. As shown, for instance, in FIG. 16C the first bristles 1644 include first scrubber cutting surfaces 1652 along the edges of the bristles 1644 and the second bristles 1648 include second scrubber cutting surfaces 1654. In one example, the second scrubber cutting surfaces 1654 are sharper and are thereby able to more easily and quickly remove thrombus from the vessel wall. In combination with the shorter length of the second bristles 1648 the second scrubbing assembly 1608 is able to easily reciprocate within the vessel and correspondingly cut and abrade thrombus from the vessel wall in a rapid fashion while the first bristles 1644 atraumatically engage with the vessel wall, anchor the catheter assembly 1600 in place within the vessel, and in at least one example provide a filter to substantially prevent the egress of loose thrombus beyond the treatment area provided by the catheter assembly 1600 (e.g., the first deployed scrubbing assembly 1606).

In one example, the enhanced second scrubber cutting surfaces 1654 of the deformable strut 1614 are provided through one or more of material selection, machining of the cuts into the catheter body to form the deformable struts 1614, and mechanical or chemical processing of the deformable struts 1614. In at least some examples the first and second scrubbing assemblies 1606, 1608 are constructed with differing materials configured to provide the mechanical properties necessary for operation of the catheter assembly 1600 as described herein. For instance, in one example, the first scrubbing assembly 1606 including the deformable struts 1602 are constructed with, but not limited to, deformable softer materials including some polymers, metals and the like. In another example, the second scrubbing assembly 1608 including the plurality of deformable struts 1614 is constructed with a more rigid material capable of similarly providing a sharper second scrubber cutting surface 1654 to each or one or more of the deformable struts 1614. In one example, the second scrubber cutting surfaces 1654 and the deformable struts 1614 are constructed with but not limited to stainless steel, Nitinol and the like and include a sharper cutting surface relative to the cutting surfaces (e.g., blunt surfaces) of the first scrubbing assembly 1606.

While the catheter assembly 1600 is operated to remove the thrombus from the vessel, for instance through reciprocation of the second scrubbing catheter 1604 relative to the first scrubbing catheter 1602, in one example, an aspiration catheter is optionally provided with the catheter assembly 1600. The aspiration catheter includes an aspiration orifice sized and shaped to remove loose thrombus from the treatment location and thereby substantially prevent egress of loose thrombus past the screen provided by the first scrubbing assembly 1606 in the deployed configuration shown in FIG. 16C.

In yet another example, the catheter assembly 1600 is used in combination with the thrombectomy catheter 700 as previously described herein. For instance, the thrombectomy catheter 700 including the circular flow 718 provided by the inflow and outflow orifices 712, 714 cooperates with the mechanical abrading function of one or both of the first and second scrubbing assemblies 1606, 1608 to provide a composite hydrodynamic and mechanical thrombectomy procedure that maximizes the removal of thrombus from the treatment location. In another example, the thrombectomy catheter 700 is provided to withdraw thrombus removed by the reciprocation of one or both of the first and second scrubbing catheters 1602, 1604. In this example, the circular flow 718 from the thrombectomy catheter 700 as well as the aspiration provided at the distal opening 716 is used to aspirate loose thrombus particles from the treatment location caused by the mechanical abrading developed through reciprocation of the second scrubbing assembly 1608. In this example, the circular flow 718 provided by the thrombectomy catheter 700 is optionally used to remove thrombus in combination with the second scrubbing assembly 1608.

### CONCLUSION

The catheter assemblies including the deployable scrubber assemblies described herein provide brush assemblies including one or more bristles configured for sweeping engagement and abrasion of organized thrombus within vessels. The deployable scrubber assemblies provide a plurality of bristles formed with a plurality of deformable struts having strut bases moved into close engagement as components of engaged proximal and distal shoulders. The shoulders including the strut bases form structural supports for each of the bristles. Bristles extend radially away from the catheter assembly and thereby provide one or more layers of scrubbing bristles configured for engagement and abrading of organized thrombus along the vessel wall. Moreover, the deployable scrubber assembly is actively positioned in the deployed configuration through movement of the inner mandrel that engages the shoulders and forms the structural base for the bristles. In contrast, passive cage assemblies use shape memory materials and the like to deploy cages that are readily collapsed when engaged with a vessel wall, thrombus and the like. By actively, deploying the scrubber assembly and retaining the deployment through engagement of the shoulders undesired collapse of the scrubber bristles is avoided.

As previously shown the bristles in one example are formed into one or more two dimensional shapes that are capable of reciprocating and removing thrombus without collapsing toward the catheter body. Because the bristles are organized into two dimensional shapes radial collapse of the bristles is substantially prevented (as is the case with three dimensional cages having opposed spaced apart bases). In one example, the shoulders including the strut bases are engaged in a deployed configuration to provide stable structural support and facilitate transmission of reciprocating movement to the bristles for abrading of organized thrombus. The bristles, as previously described sweep back and forth along thrombus (and deflect in a manner similar to brush bristles) to abrade and remove thrombus without allowing collapse of the bristles into a configuration such as the stored configuration.

Further, the plurality of deformable struts forming the bristles used in the deployable scrubber assembly are in one example formed from a single tube that includes the proximal and distal shoulders. For instance, the proximal and distal shoulders as well as the plurality of deformable struts are formed from a single tube and a plurality of cuts or slots are formed in the single tube and provide channels that facilitate the bending and deflection of the plurality of deformable struts into the bristles shown herein. By changing features of the struts, such as the angles of cuts and the thickness of the plurality of deformable struts varying configurations and shapes of the bristles are provided when in the deployed configuration. Further, in some examples the cuts or slots in the single tube are formed in such a manner that nodes are provided between one or more of the plurality of deformable struts between the proximal and distal shoulders. The nodes form corresponding cells within the deployed scrubber assembly and more tightly pack the cutting surfaces of the bristles to facilitate enhanced abrading of organized thrombus within a vessel.

In the foregoing description, the subject matter has been described with reference to specific exemplary examples. However, it will be appreciated that various modifications and changes may be made without departing from the scope of the present subject matter as set forth herein. The description and figures are to be regarded in an illustrative manner, rather than a restrictive one and all such modifications are intended to be included within the scope of the present subject matter. Accordingly, the scope of the subject matter should be determined by the generic examples described herein and their legal equivalents rather than by merely the specific examples described above. For example, the steps recited in any method or process example may be executed in any order and are not limited to the explicit order presented in the specific examples. Additionally, the components and/or elements recited in any apparatus example may be assembled or otherwise operationally configured in a variety of permutations to produce substantially the same result as the present subject matter and are accordingly not limited to the specific configuration recited in the specific examples.

Benefits, other advantages and solutions to problems have been described above with regard to particular examples; however, any benefit, advantage, solution to problems or any element that may cause any particular benefit, advantage or solution to occur or to become more pronounced are not to be construed as critical, required or essential features or components.

The present subject matter has been described above with reference to examples. However, changes and modifications may be made to the examples without departing from the scope of the present subject matter. These and other changes or modifications are intended to be included within the scope of the present subject matter, as expressed in the following claims.

It is to be understood that the above description is intended to be illustrative, and not restrictive. Many other examples will be apparent to those of skill in the art upon reading and understanding the above description. It should be noted that examples discussed in different portions of the description or referred to in different drawings can be combined to form additional examples of the present application. The scope of the subject matter should, therefore, be determined with reference to the appended claims, along with the full scope of equivalents to which such claims are entitled.

1. A catheter assembly comprising:
an outer sheath;
an inner mandrel slidably coupled within the outer sheath; and
a scrubbing assembly coupled between the inner mandrel and the outer sheath, the scrubbing assembly including:
a plurality of deformable struts deflectable according to movement of the inner mandrel relative to the outer sheath,
a proximal shoulder coupled with the outer sheath,
a distal shoulder coupled with the inner mandrel, and the plurality of struts extend between the proximal and distal shoulders, and
the scrubbing assembly is movable between a stored configuration and a deployed configuration according to movement of the inner mandrel relative to the outer sheath:
in the stored configuration the proximal and distal shoulders are remote from each other, and
in the deployed configuration the proximal shoulder is engaged with the distal shoulder, and the deformable struts are deflected into bristles.

2. The catheter assembly of claim 1, wherein the proximal and distal shoulders include respective proximal and distal strut bases, and the proximal and distal shoulders are engaged in the deployed configuration through one or more of the proximal and distal strut bases.

3. The catheter assembly of claim 2, wherein the plurality of deformable struts extend from the proximal and distal strut bases.

4. The catheter assembly of claim 1, wherein in the deployed configuration, the deformable struts are planar bristles.

5. The catheter assembly of claim 1, wherein the deformable struts include tips in the deployed configuration, and the tips overlie the engaged proximal and distal shoulders.

6. The catheter assembly of claim 1 comprising a second plurality of deformable struts and corresponding second proximal and second distal shoulders coupled with the second plurality of deformable struts,
the second proximal shoulder is coupled with the distal shoulder, and the second distal shoulder is coupled with the inner mandrel, and
in the deployed configuration the second plurality of deformable struts are deflected into bristles separate from the bristles of the plurality of deformable struts.

7. (canceled)

8. The catheter assembly of claim 6 comprising an intermediate shoulder and the intermediate shoulder includes the distal shoulder and the second proximal shoulder or the second distal shoulder and the proximal shoulder, and the intermediate shoulder is slidably coupled with the inner mandrel.

9. The catheter assembly of claim 1, wherein one or more of the plurality of deformable struts are engaged with one or more of the plurality of deformable struts at nodes, and in the deployed configuration the bristles include open cells extending between one or more of the nodes or the proximal and distal shoulders.

10. The catheter assembly of claim 1, wherein a single tube includes the plurality of deformable struts and the proximal and distal shoulders, the single tube includes a plurality of cuts extending between the proximal and distal shoulders, and the plurality of deformable struts extend along the plurality of cuts.

11. (canceled)

12. (canceled)

13. (canceled)

14. (canceled)

15. (canceled)

16. (canceled)

17. (canceled)

18. (canceled)

19. (canceled)

20. (canceled)

21. A thrombectomy catheter assembly comprising:
a thrombectomy catheter including:
a catheter body extending from a catheter proximal portion to a catheter distal portion;
a fluid jet emanator within a catheter lumen near the catheter distal portion;
an inflow orifice extending from the catheter lumen through a catheter sidewall, the inflow orifice is adjacent to the fluid jet emanator;
an outflow orifice extending from the catheter lumen through the catheter sidewall, the outflow orifice is remotely positioned from the fluid jet emanator relative to the inflow orifice;
a scrubbing system slidably coupled within the catheter lumen, the scrubbing system includes:
an inner mandrel;
an outer sheath slidably coupled along the inner mandrel and slidably coupled with the catheter body; and
a scrubbing assembly including a plurality of deformable struts coupled between the inner mandrel and the outer sheath at proximal and distal shoulders, the scrubbing assembly is movable between a stored configuration and a deployed configuration according to movement of the inner mandrel relative to the outer sheath:
in the stored configuration the deformable struts lie along the inner mandrel and the proximal and distal shoulder are remote from each other, and
in the deployed configuration the proximal shoulder is engaged with the distal shoulder, and the deformable struts are deflected into a planar bristle configuration.

22. (canceled)

23. (canceled)

24. The thrombectomy catheter assembly of claim 21, wherein the proximal shoulder is coupled with the outer sheath, and the distal shoulder is coupled with the inner mandrel.

25. (canceled)

26. The thrombectomy catheter assembly of claim 21 comprising a second plurality of deformable struts and corresponding second proximal and second distal shoulders coupled with the second plurality of deformable struts,
the second proximal shoulder is coupled with the distal shoulder, and the second distal shoulder is coupled with the inner mandrel, and
in the deployed configuration the second plurality of deformable struts are deflected into a second planar bristle configuration separate from the planar bristle configuration.

27. (canceled)

28. (canceled)

29. (canceled)

30. A catheter assembly comprising:
a first scrubbing catheter including a first scrubbing assembly near a first catheter distal portion;
a second scrubbing catheter including a second scrubbing assembly near a second catheter distal portion, the first scrubbing catheter is slidably received within a second catheter lumen of the second scrubbing catheter;
each of the first and second scrubbing assemblies includes:
a plurality of deformable struts,
a proximal shoulder coupled at a proximal portion of the deformable struts,
a distal shoulder coupled at a distal portion of the deformable struts; and
each of the first and second scrubbing assemblies is separately movable between a stored configuration and a deployed configuration according to movement of the respective proximal shoulder relative to the respective distal shoulder:
in the stored configuration the proximal and distal shoulders are remote from each other, and
in the deployed configuration the proximal shoulder is engaged with the distal shoulder, and the deformable struts are deflected into bristles.

31. The catheter assembly of claim 30, wherein the first scrubbing catheter includes a first outer sheath and a first inner mandrel, and the first outer sheath is coupled with the proximal shoulder of the first scrubber assembly and the first inner mandrel is coupled with the distal shoulder of the first scrubber assembly, and
The second scrubbing catheter includes a second outer sheath and a second inner mandrel, the first outer sheath is slidably received within the second outer sheath, and the second outer sheath is coupled with the proximal shoulder of the second scrubber assembly and the second inner mandrel is coupled with the distal shoulder of the second scrubber assembly.

32. The catheter assembly of claim 30, wherein the first scrubber assembly has a greater width than the second scrubber assembly while both the first and second scrubber assemblies are in the deployed configuration.

33. (canceled)

34. The catheter assembly of claim 30, wherein the deformable struts of the second scrubber assembly are more rigid than the deformable struts of the first scrubber assembly.

35. The catheter assembly of claim 30, wherein the deformable struts of the first scrubber assembly include one or more first scrubber cutting surfaces and the deformable struts of the second scrubber assembly include one or more second scrubber cutting surfaces sharper than the first scrubber cutting surfaces.

36. (canceled)

37. The catheter assembly of claim 30, wherein the thrombectomy catheter includes:
a catheter body extending from a thrombectomy catheter proximal portion to a thrombectomy catheter distal portion;
a fluid jet emanator within a thrombectomy catheter lumen near the thrombectomy catheter distal portion;
an inflow orifice extending from the thrombectomy catheter lumen through a catheter sidewall, the inflow orifice is adjacent to the fluid jet emanator; and
an outflow orifice extending from the catheter lumen through the catheter sidewall, the outflow orifice is remotely positioned from the fluid jet emanator relative to the inflow orifice.

38. The catheter assembly of claim 30, wherein the thrombectomy catheter includes a distal aspiration opening near a thrombectomy catheter distal portion.

39. The catheter assembly of claim 30 comprising an aspiration catheter, and the second scrubbing catheter is received within an aspiration catheter lumen of the aspiration catheter.
